(19) United States
(12) Patent Application Publication
(10) Pub. No.: US 2021/0220270 A1
(43) Pub. Date: Jul. 22, 2021

(54) Composition

(71) Applicant: Maria Francesca CORDEIRO, Bray Berks (GB)

(72) Inventors: Maria Francesca Cordeiro, London (GB); Benjamin Michael Davis, East Hagbourne (GB); Satyanarayana Somavarapu, London (GB)

(21) Appl. No.: 15/734,057

(22) PCT Filed: Jun. 27, 2019
(86) PCT No.: PCT/GB2019/051812

§ 371 Date: Dec. 1, 2020

(30) Foreign Application Priority Data
Jun. 27, 2018 (GB) 1810492.7

(51) Int. Cl. A61K 9/107; A61K 31/426; A61K 31/05; A61K 31/12; A61K 9/00

(52) U.S. Cl. CPC A61K 9/1075; A61K 31/426; A61K 31/05; B82Y 5/00; A61K 9/0043; A61K 9/0048; A61K 31/12

(57) ABSTRACT

The present invention provides a pharmaceutical composition comprising a peroxisome proliferator activated receptor (PPAR) modulator and an polymeric nanocarrier component, wherein the polymeric nanocarrier component is capable of solubilising the PPAR modulator in an aqueous medium and, wherein in the polymeric nanocarrier component is a micelle forming non-ionic surfactant. Uses of the same in therapy are also provided.

### FIELD OF INVENTION

The present invention relates to novel formulations of peroxisome proliferator activated receptor (PPAR) modulators and uses of the same in therapy. It also relates to the use of acylhomoserine lactones (PPAR modulators) for enhancing drug delivery via nonparenteral or topical administration routes.

### BACKGROUND TO THE INVENTION

The present invention is concerned, in one aspect, with the treatment or prevention of neurodegenerative conditions, retinal disorders, and brain disorders, as well as pulmonary arterial hypertension, cancer and antifibrotic disorders. Neurodegenerative conditions affect various parts of the central and peripheral nervous systems, and include Parkinson's Disease, Alzheimer's Disease and Huntington's Disease. Retinal disorders may include retinal degenerative conditions such as glaucoma, age-related macular degeneration (AMD), diabetic retinopathy and optic neuritis. Brain disorders may include traumatic brain injury, stroke, cerebral palsy, e.g. as caused by neonatal hypoxia, and cancer, including brain tumours.

Peroxisome proliferator-activated receptors (PPARs) are ligand-activated transcription factors belonging to the superfamily of nuclear hormonal receptors. These receptors were identified in the hepatocytes of rodents in 1990, and the name comes from their ability to induce peroxisome proliferation. PPARs interact directly with PPAR gamma coactivators 1-alpha (PGC-1a) and 1-beta (PGC-1β) in the regulation of mitochondrial biogenesis, through the detection and control of lipid homeostasis. These receptors also regulate the expression of genes coding for uncoupling proteins (UCPs). UCPs are transporters in the inner mitochondrial membrane involved in the control of thermogenesis, ROS production, and oxidative function. By binding to a specific sequence in the promoter region of target genes, PPARs are able to regulate gene transcription. Activated PPARs can also directly inhibit transcription factors. These functions allow PPARs to encourage lipid consumption in the production of ATP when there is a cellular demand for energy. Many PPAR modulators have poor aqueous solubility and/or bioactivity which may limit therapeutic utility.

Rosiglitazone is an exogenous agonist of PPAR-gamma and belongs to the thiazolidinedione family, which acts as insulin sensitizers. Rosiglitazone was originally used to counter insulin resistance in type 2 diabetes, has recently shown promise as a therapy in animal models of PD (Normando et al 2016). Rosiglitazone therapy is reported to promote an anti-inflammatory response, with attenuation of microglial activation, release of pro-inflammatory cytokines, oxidative stress, astrocytic gliosis and reversible inhibition of monoamine oxidase—a crucial enzyme for dopamine metabolism. The outcomes of clinical investigations of thiazolidinedione therapies for the treatment of PD have so far been complex and administration of these agents is reported to not slow disease progression.

More recently, curcumin, resveratrol and acylhomoserine lactones (AHLs) have each been reported to bind PPARs and this interaction has been found to contribute to their biological activity and/or therapeutic action, including: promotion of an anti-inflammatory response, with attenuation of microglial activation, release of pro-inflammatory cytokines and oxidative stress. AHLs are documented to compete with RSG binding to the same site on PPAR-gamma (Jahoor et al 2008; Cooley et al 2010). Like other PPAR modulators, these compounds suffer from poor aqueous solubility and/or bioavailability.

There is hence a need to provide improved formulations for PPAR modulators in order to improve bioavailability and/or bioactivity.

### SUMMARY OF THE INVENTION

In a first aspect the present invention provides a pharmaceutical composition comprising a peroxisome proliferator activated receptor (PPAR) modulator incorporated within a polymeric nanocarrier component, wherein the polymeric nanocarrier component is capable of solubilising the PPAR modulators in an aqueous medium. PPAR modulators are known to have low solubility in water, requiring the use of solvents such as dimethyl sulfoxide (DMSO). However, the present inventors have found that polymeric nanocarriers can be used to solubilise PPAR modulators at physiological pH (about pH 4 to about pH 8). The compositions of the invention can therefore be prepared without the need to use potentially harmful solvents such as dimethyl sulfoxide (DMSO). Surprisingly, compositions of the invention have been observed to have neuroprotective effects in in vivo models of Central Nervous System Injury. Additionally, systemic administration of such compositions was observed to have a neuroprotective effect on the retina and CNS. Compositions of the invention have been shown to exhibit greater neuroprotective efficacy than administration of PPAR modulators alone.

The PPAR modulator may be an endogenous or exogenous molecule and includes compounds which modulate the activity of a PPAR such that net receptor activity is changed, i.e. the compounds may act as PPAR agonists or inhibitors. For example, activity of a PPAR may be modulated by PPAR agonists binding to the receptor or acting on downstream components of the pathway activated by the PPAR to induce similar activity. PPAR agonists may be superagonists, partial agonists or full agonists. PPAR modulators may have a binding affinity for the PPAR of 100 μM or less, preferably 10 μM or less, 5 μM or less, or more preferably 2 μM or less or 1 μM or less. In embodiments of the invention PPAR modulators may have a binding affinity for the PPAR of 100 nM or less or 10 nM or less.

The polymeric nanocarrier component as used herein refers to a component comprising a polymer. For example, the polymeric nanocarrier component may comprise a polyethylene glycol (PEG) group and/or a polymer based constituent, such as a poloxamer. Preferably the polymeric nanocarrier component is a surfactant or a synthetic derivative thereof.

The polymeric nanocarrier component may be a non-ionic surfactant and/or may be a micelle forming surfactant. Advantageously, non-ionic micelle forming surfactants form relatively soft/flexible micelles, which can enhance their transport across non-parenteral routes of administration, such as across mucosal membranes or biological membranes. Non-ionic surfactants may include polysorbates (Tweens), Triton X-100, polyethoxylated castor oil, and Solutol HS. In embodiments of the invention the micelle forming surfactant may be selected from one or more of D-α-tocopherol polyethylene glycol 1000 succinate (Vitamin E TPGS), PEGylated phospholipid derivatives (e.g. DSPE-PEG, DSPS-PEG etc.), poloxamers (e.g. Lutrol F68, Lutrol F127 etc.), poly(lactic-co-glycolic acid) (PLGA) or chitosan derivatives. Optionally, PEG derivatives may be further functionalised via addition of a “click-chemistry” reactive group such as maleimide-PEG for covalent conjugation to thiol groups or azide-PEG/alkyne-PEG for covalent conjugation to alkyne/azide functionalised targeting moieties, e.g. TPGS-PEG-MAL, DSPE-PEG-AZIDE etc. Said functionalised targeting moieties may include proteins or peptides, including phosphatidylserine binding proteins such as annexins, especially annexin V or functional fragments or functional derivatives thereof, which preferably comprise the annexin repeat. Alternatively, His-tagged proteins or peptides can be non-covalently associated with the particles surface using Nickel functionalised lipids (e.g. 18:1 DGS-NTA(Ni)).

In preferred embodiments of the invention the polymeric nanocarrier component comprises Vitamin E TPGS, optionally in combination with Lutrol F127, Solutol HS, chitosan or DSPE-PEG. Vitamin E TPGS is a non-ionic surfactant that forms stable micelles at concentrations of greater than 0.02% w/w, providing a low critical micelle concentration. The α-tocopherol component also has an endogenous nature and antioxidant properties, as well as P-glycoprotein antagonism, which can enhance the barrier crossing ability of formulations containing this agent. Polymeric nanocarrier compositions of the present invention may comprise Vitamin E TPGS at concentrations of about 0.02 mg/mL to about 100 mg/mL, preferably about 10 mg/mL to about 65 mg/mL, more preferably about 20 to about 55 mg/mL.

Lutrol F127 is a difunctional block copolymer surfactant consisting of a central hydrophobic polyoxypropylene group flanked by hydrophilic polyoxyethylene groups, and can sterically stabilise nanocarriers against aggregation. Polymeric nanocarrier compositions of the present invention may comprise Lutrol F127 at concentrations of about 0.2% w/v to about 30% w/v, preferably about 5% w/v to about 20% w/v, more preferably about 10% w/v to about 20% w/v. Lutrol F127 may be used alone or in combination with Vitamin E TPGS.

Solutol HS (2-hydroxyethyl 12-hydroxyoctadecanoate) is non-ionic solubilizer and emulsifying agent, with low toxicity. Polymeric nanocarrier compositions of the present invention may comprise Solutol HS at concentrations of about 100 mg/mL to about 200 mg/mL. Solutol HS is preferably used in combination with Vitamin E TPGS.

The polymeric nanocarrier component may be up to 100% micelle forming surfactant, for example, the polymer nanocarrier component may be up to 100% vitamin E TPGS or up to 100% Lutrol F127. Alternatively, the micelle forming surfactant may comprise a combination of surfactants, such as Vitamin E TPGS in combination with a PEGylated phospholipid derivative (e.g. DSPE-PEG), a non-ionic surfactant (e.g. Solutol HS) or a poloxamer (e.g. Lutrol F127).

The composition is preferably in the form of an encapsulated formulation, most preferably a micelle. Without being bound by theory, the inventors believe that encapsulation of the PPAR modulator may improve bioavailability of this component by providing sustained release of the PPAR modulator and protect PPAR modulators against hydrolytic degradation. Compositions of the invention have been observed to exhibit a greater neuroprotective effect than PPAR modulators administered in an unencapsulated form.

Micellar nanocarriers of the present invention can have a diameter of about 100 nm or less, preferably about 70 nm or less or about 50 nm or less. In preferred embodiments of the invention micellar nanocarriers have a diameter of about 30 nm or less. Micellar nanocarriers may have a minimum diameter of about 10 nm. Preferably the micellar nanocarriers have a diameter of about 20 nm. Liposomes on the absence of sizing (e.g. extrusion process) are heterogeneous in size, often ranging from 100 nm to 1000 nm in diameter. In contrast, micellar nanocarriers of the present invention are substantially homogenous in diameter. For example, at least 70% or at least 80% or at least 90% of the micellar nanocarriers in a composition of the present invention may have a diameter between about 10 nm and about 30 nm.

Solubilisation of the PPAR modulator preferably refers to encapsulation of the PPAR modulator, which may be quantified by encapsulation efficiency. When carried out at physiological pH encapsulation efficiency is preferably at least 5% or at least 10% or at least 15%. In embodiments of the invention encapsulation efficiency may be at least 20% or at least 25%. In embodiments of the invention encapsulation efficiency may be 50% or more, or 70% or more, or 80% or more and may be up to 100%.

Encapsulated formulations may comprise the PPAR modulator at concentrations of from about 0.1 mg/mL to about 100 mg/mL, preferably about 0.5 mg/mL to about 50 mg/mL, more preferably about 1 mg/mL to about 10 mg/mL.

In preferred embodiments of the invention the composition is in the form of a ternary system comprising an aqueous continuous phase, the PPAR modulator and polymeric nanocarrier component being predominantly present in a disperse phase distributed therein.

The PPAR modulator may be a PPAR-alpha modulator, a PPAR-gamma modulator, a PPAR-delta modulator, a dual PPAR modulator or a pan PPAR modulator. In preferred embodiments of the invention the PPAR modulator is a PPAR-gamma agonist or a compound having PPAR-gamma agonist activity. Without being bound by theory, the present inventors believe that PPAR-gamma agonists (or compounds having such activity) act on neurons and retinal ganglion cells (RGCs) to mitigate oxidative stress, reduce microglia activation and pro-inflammatory cytokine release and promote mitochondrial biogenesis. These pathways have been implicated in the pathogenesis of certain CNS disorders, including glaucoma and Parkinson's Disease, suggesting a mechanism of action in the treatment of such disorders as described herein.

The PPAR agonist may be a thiazolidinedione. In embodiments of the invention the PPAR agonist may be selected from one or more of pioglitazone, rosiglitazone, lobeglitazone, ciglitazone, darglitazone, englitazone, netoglitazone, rivoglitazone, Phytocannabinoid 49-THCA and troglitazone.

Alternatively, as mentioned above, the PPAR modulator may be a compound that modulates the activity of a PPAR such that net receptor activity is increased, i.e. a compound that has the same net effect as an agonist. Such compounds include curcumin and resveratrol, which are known to have PPAR-gamma activity.

Alternatively, the PPAR modulator may be an acylhomoserine lactone (AHSL) compound. Such compounds have been shown to have PPAR modulation capability (Jahoor et al.). The AHSL may, for example, have an acyl group of 4 to 20 carbons in length. 3-oxo and 3-hydroxy derivatives may also be mentioned, as may the tetramic acid and tetronic acid derivatives of AHSLs. Exemplary AHSL compounds include 3-hydroxydodecanoyl homoserine lactone and 3-oxododecanoyl homoserine lactone. AHSL compounds have the additional advantage that they interact with tight junctions to increase permeability of biological barriers (Karlsson et al.). This can enhance delivery of the composition to the intended tissue in vivo.

For example, suitable polymeric nanocarrier compositions of the present invention may be micellar formulations of:
about 10 mg/ml vitamin E TPGS
about 20 mg/ml Lutrol F127, and
about 5 mg/ml curcumin; or
about 25 mg/ml vitamin E TPGS
about 150 mg/ml Solutol HS and
about 5 mg/ml curcumin, or about 15 mg/ml resveratrol; or
about 50 mg/ml vitamin E TPGS
about 150 mg/ml Solutol HS
about 5 mg/ml curcumin, or about 15 mg/ml resveratrol.

Compositions of the invention may comprise combinations of two or more PPAR modulators. Preferably at least one of the PPAR modulators is encapsulated as described above. Alternatively, both PPAR modulators may be encapsulated. When two or more PPAR modulators are encapsulated the type of encapsulation may be the same or different. For example, both PPAR modulators may be encapsulated in a single polymeric nanocarrier, or each PPAR modulator may be encapsulated in a separate polymeric nanocarrier which are combine prior to administration. In embodiments of the invention the composition may comprise a combination of resveratrol and curcumin.

The composition may be sterile and may comprise one or more pharmaceutically acceptable carriers or excipients. Suitable carriers and excipients will be familiar to the skilled person and may be optimised in line with the intended route of delivery. For example, compositions of the inventions may include buffers, binders, preservatives, thickeners or antioxidants, such as trehalose.

Preferably the composition is suitable for topical delivery, including ocular and nasal delivery, oral or dermal delivery. Local delivery of the composition may be advantageous due to reducing systemic exposure to the PPAR modulator, which have been linked to harmful side effects including increased risk of myocardial infarction and death.

Topical formulations are preferably in the form of a solution or suspension in an aqueous medium, such as a solution, lotion, gel, cream, ointment, gel or foam. Oral formulations may be in the form of solutions, suspensions, tablets, capsules, powders or granules. Parenteral administration may include intravenous, subcutaneous or intraperitoneal administration. Parenteral formulations in particular may be in the form of solutions or suspensions in aqueous media or may be provided as a lyophilised powder.

In embodiments of the invention the composition is suitable for intranasal delivery. Such formulations may be in the form of a solution, suspension or dry powder suitable for inhalation.

In general, and especially where the composition is to be administered in liquid form (via any of the above routes), the composition of the invention may be provided as a lyophilised powder. The compositions of the invention have been determined to be stable to lyophilisation, and can be reconstituted using, for example, normal saline or other (preferably aqueous) vehicles. It is preferred, when lyophilisation is to take place, to include a cryoprotectant material such as trehalose in the composition.

The composition of the invention may be used in therapy. In particular, the composition of the invention may be used in the treatment or prevention of a CNS disorder, such as a neurodegenerative disorder, a retinal disorder or a brain disorder.

In a further aspect the present invention provides a method for treating a CNS disorder, such as a neurodegenerative disorder, a retinal disorder or a brain disorder, the method comprising administering a composition of the invention to a patient. The patient is preferably a mammal, including a human, and may be a paediatric or geriatric patient.

The neurodegenerative condition may be Parkinson's Disease, Alzheimer's Disease or Huntington's Disease. Retinal disorders may include retinal degenerative conditions such as glaucoma, age-related macular degeneration (AMD), diabetic retinopathy and optic neuritis. Brain disorders may include traumatic brain injury, stroke, cerebral palsy, e.g. as caused by neonatal hypoxia, and cancer, including brain tumours. In embodiments of the invention the composition may be for use in the treatment of pre-symptomatic Parkinson's Disease.

The composition may be administered topically, such as ocularly, or intranasally, as described above. In embodiments of the invention the composition may be administered in combination with one or more additional therapeutic agents, such as insulin, metformin, dipeptidyl peptidase-4 (DPP-4) inhibitors (such as Alogliptin), glucagon-like peptide-1 (GLP-1) receptor agonists, antioxidants (such as resveratrol, Coenzyme Q10, Idebenone, Quercetin etc.), compounds of the vitamin D group, or derivatives thereof, vascular endothelial growth factor (VEGF) antagonists (such as ranibizumab, bevacizumab or functional fragments thereof), N-methyl-D-aspartate (NMDA) receptor antagonists, glutamate antagonists or memantine. The additional therapeutic agent may be administered simultaneously with the composition of the invention or may be administered sequentially. Where the additional therapeutic agent is administered simultaneously with the composition of the invention, it may be included in the composition of the invention, either in the same phase as the PPAR or in the continuous phase of a ternary composition. In a particular embodiment, the addition therapeutic agent has a hydrophobicity such that both it and the PPAR are present in the disperse phase (i.e. co-encapsulated). In embodiments of the invention the composition comprises curcumin and an antioxidant, such as resveratrol.

In a further aspect the present invention provides a method for preparing a PPAR modulator micellar composition as described above, the method comprising: (i) dissolving one or more polymeric nanocarrier components in a first solvent mixture; (ii) dissolving a PPAR modulator in a second solvent mixture; (iii) combining the dissolved polymeric nanocarrier component and dissolved PPAR modulator and drying the combination to a form a film; (iv) rehydrating the film with buffer to form a micelle solution; (v) filtering the suspension to remove unencapsulated PPAR modulator. Preferably, the first solvent mixture is a short chain primary alcohol, such as ethanol. Compared to other solvents such as chloroform/methanol, ethanol is less toxic, meaning that residual solvent which may be present in the composition is unlikely to be problematic. In embodiments of the invention the first and second solvent mixtures may be the same. In preferred embodiments of the method the PPAR modulator is curcumin, resveratrol or AHSL, which have been shown to be poorly soluble in other solvent mixtures. Preferably the suspension is filtered through a membrane filter having a pore size of about 0.22 μm, which can additionally remove any potential biological contaminants.

In an additional aspect the present invention provides a pharmaceutical composition comprising an active pharmaceutical ingredient (API) and an AHSL compound. The AHSL may, for example, have an acyl group of 4 to 20 carbons in length. 3-oxo and 3-hydroxy derivatives may also be included, as may the tetramic acid and tetronic acid derivatives of AHSLs. Exemplary AHSL compounds include 3-hydroxydodecanoyl homoserine lactone and 3-oxododecanoyl homoserine lactone.

As explained above, AHSL compounds interact with tight junctions to increase permeability of biological barriers. These compounds are known to be used by certain bacteria as part of the tissue invasion process during the establishment of an infection of a host. However, the potential of these compounds as a means for delivering an API into target tissues has not been recognised previously, and the present inventors have determined that such an approach may be used with a large range of APIs, including aforementioned PPAR modulators (curcumin, resveratrol etc) and those with high molecular weights, such as peptides or antibodies.

In a preferred embodiment, the composition of this aspect further includes an polymeric nanocarrier component encapsulating the AHSL and/or the API in liposomes or micelles. The polymeric nanocarrier component is preferably in the form of a liposome, which may include one or more phospholipids and can also include a sterol such as cholesterol and/or a vitamin E derivative such as TPGS. Suitable phospholipids include, for example, those based on phosphatidylcholine, phosphatidylserine and phosphatidylethanolomine. In more detail, phospholipids for use in compositions of the invention may include natural phospholipid derivatives or synthetic phospholipid derivatives. Natural phospholipid derivatives may include one or more of egg phosphatidylcholine, hydrogenated egg phosphatidylcholine, soy phosphatidylcholine, hydrogenated soy phosphatidylcholine or sphingomyelin, such as 1-Myristoyl-2-palmitoyl-sn-glycero-3-phosphocholine, 1-Myristoyl-2-stearoyl-sn-glycero-3-phosphocholine, 1-Palmitoyl-2-myristoyl-sn-glycero-3-phosphocholine, 1-Palmitoyl-2-oleoyl-sn-glycero-3-phosphocholine, 1-Palmitoyl-2-oleoyl-sn-glycero-3-phosphoethanolamine, 1-Palmitoyl-2-stearoyl-sn-glycero-3-phosphocholine, 1-Stearoyl-2-myristoyl-sn-glycero-3-phosphocholine, 1-Stearoyl-2-oleoyl-sn-glycero-3-phosphocholine and 1-Stearoyl-2-palmitoyl-sn-glycero-3-phosphocholine. Synthetic phospholipid derivatives may include one or more of 1,2-Didecanoyl-sn-glycero-3-phosphocholine (DDPC), 1,2-Dierucoyl-sn-glycero-3-phosphocholine (DEPC), 1,2-Dierucoyl-sn-glycero-3-phosphoethanolamine (DEPE), 1,2-Dilinoleoyl-sn-glycero-3-phosphocholine (DLOPC), 1,2-Dilauroyl-sn-glycero-3-phosphocholine (DLPC), 1,2-Dilauroyl-sn-glycero-3-phosphoethanolamine (DLPE), 1,2-Dilauroyl-sn-glycero-3-phosphoserine (DLPS), 1,2-Dimyristoyl-sn-glycero-3-phosphocholine (DMPC), 1,2-Dimyristoyl-sn-glycero-3-phosphoethanolamine (DMPE), 1,2-Dimyristoyl-sn-glycero-3-phosphoserine (DMPS), 1,2-Dioleoyl-sn-glycero-3-phosphocholine (DOPC), 1,2-Dioleoyl-sn-glycero-3-phosphoethanolamine (DOPE), 1,2-Dioleoyl-sn-glycero-3-phosphoserine (DOPS), 1,2-Dipalmitoyl-sn-glycero-3-phosphocholine (DPPC), 1,2-Dipalmitoyl-sn-glycero-3-phosphoethanolamine (DPPC), 1,2-Dipalmitoyl-sn-glycero-3-phosphoserine (DPPS), 1,2-Distearoyl-sn-glycero-3-phosphocholine (DSPC), 1,2-Distearoyl-sn-glycero-3-phosphoethanolamine (DSPE) and 1,2-Distearoyl-sn-glycero-3-phosphoserine (DSPS). In embodiments of the invention the surfactant or lipid may be conjugated to polyethylene glycol (PEG), e.g. PLGA-PEG. Alternatively, the polymeric nanocarrier component may be as defined in relation to the first aspect of the invention.

Some AHSLs are capable of forming micelle-like structures in aqueous media, and hence are able to act as a delivery system for an API without addition of further components. However, the stability of the composition can be improved by the inclusion of an polymeric nanocarrier component, e.g. as defined above.

The API to be included in the composition of this aspect is not particularly limited, and the skilled person would readily be able to determine which APIs could potentially be employed. As examples, however, the various API-types mentioned above in relation to the first aspect may be considered.

In a related aspect, the invention provides the use of an AHSL for enhancing delivery of an API. In particular, delivery across biological barriers (such as the blood-brain barrier, blood-retinal barrier etc) is enhanced.

### BRIEF DESCRIPTION OF THE DRAWINGS

The invention will now be described in detail, by way of example only, with reference to the figures.

FIG. 1 shows absorbance spectrum and molar extinction coefficient of curcumin in DMSO and molar extinction coefficient of stable and degraded curcumin in DMSO [A] Standard curve of known 0.005 mg/mL curcumin concentration dissolved in DMSO, which revealed peak absorbance (OD) at wavelength of 435 nm. [B] Gradient of curcumin concentration against log absorbance at 435 nm indicates molar extinction coefficient (c). [C] Curcumin degraded in sodium hydroxide (NaOH) 5 mg/mL solution (left) is much darker than solubilised stable micellar curcumin formulation) (right). [D] Molar extinction coefficient of degraded curcumin in NaOH and diluted in DMSO was compared to 5 mg/mL curcumin solution in ethanol diluted in DMSO. Over 72 hours of degradation, molar extinction coefficient fell from 22,462 L·mol−1·cm−1 to 2477 L·mol−1·cm−1, 0.04% that of the molar extinction coefficient of curcumin diluted in DMSO, 58,547 L·mol−1·cm−1.

FIG. 2 shows a thin-film hydration technique using vacuum-assisted rotary evaporation, which involves the addition of dissolved material to a round-bottomed flask [A.i] and evaporation of ethanol (EtOH) under vacuum and high temperature [A.ii], to leave a dry thin-film [A.iii]. This is resuspended in aqueous buffer to generate curcumin micelles [A.iv]. Photographic images of rotary evaporation [B.i] and remaining thin-film [B.ii].

FIG. 3 shows solubilisation of curcumin in micellar formulation 2 mg/mL curcumin is very poorly soluble in distilled water (A.i), forming a precipitate which is removed on filtration (B.i) using a 0.22 μm pore membrane to leave water (A.ii). 2 mg/mL solution of curcumin loaded micelles (A.iii) which has been passed through a 0.22 μm pore membrane filter (B.ii) showing good retention of curcumin.

FIG. 4 shows curcumin stability assessment measuring encapsulation efficiency in different environments [A] Comparison between 25° C. and 4° C. samples. Downward drop in EE for 4° C. samples compared to baseline from 3 weeks onwards [B] Comparison between 25° C. and lyophilised (25° C.) samples. At all-time points there was a significant drop in EE of lyophilised samples. Data expressed as mean with SEM. Data analysed using 2way ANOVA with Bonferroni post hoc analysis,***p<0.001

FIG. 5 shows Z-diameter and PDI of curcumin formulations over time [A] Size of micelles over time (z-diameter) stayed uniform with no significant change seen [B] Spread of micelles (PDI) did increase with time but this change was not significant. Data expressed as mean with SEM. Data analysed using two-way ANOVA with Bonferroni post hoc analysis.

FIG. 6 shows discoloration of resveratrol formulation (configuration B) over two-week period Pictures [A]-[E] correspond to time points 0, 7, 8, 9 and 14 days. The initial formulation [A] was clear without any sediment formation; this formulation was kept at room temperature in a dry, dark environment for two weeks. Within a week [B] the discolouration was observed with the formulation continuing to darken. By two weeks [E] the formulation was at its darkest however with no sediment formation.

FIG. 7 shows resveratrol formulations (Configuration C) over 9 day period, stored in different environments Pictures [A]-[E] correspond to time points 0, 2, 3, 8 and 9 days with [i] formulation kept at room temperature (25° C.) and [ii] formulation kept at 4° C. The formulations were kept in a dry and dark environment during this time. At 25° C. and 4° C. no decolouration or sedimentation was seen over the nine day period.

FIG. 8 shows resveratrol stability assessment measuring encapsulation efficiency in different environments [A] Comparison between 25° C. and 4° C. samples [B] Comparison between 25° C. and lyophilised (25° C.) samples. All samples showed a significant difference compared to baseline at each time point. [B] also indicates there was a significant difference between the two samples at 6 and 9 week time points. Data expressed as mean with SEM. Data analysed using two-way ANOVA with Bonferroni post hoc analysis, *p<0.05**p<0.01.

FIG. 9 shows Z-diameter and PDI of Resveratrol formulations over time [A] Size of micelles over time (z-diameter) stayed uniform with no significant change seen [B] Spread of micelles (PDI) stayed uniform with no significant change seen. Data expressed as mean with SEM. Data analysed using two-way ANOVA with Bonferroni post hoc analysis

FIG. 10 shows in vitro pretreatment of primary RGCs (murine) with 50 μM curcumin micelle formulation or equivalent concentrations of vehicle only, prior to paraquat treatment to induce an oxidative stress insult. Cell viability was assessed using alamarBlue assay. [A] Dose response curve; with increasing paraquat concentration, cell viability decreases. Significant increase in cell viability in curcumin pretreated cells. [B] IC50 in curcumin-treated RGCs double that of vehicle treated RGCs.

FIG. 11 shows RGC global density of treatment groups for whole retina pONT—Partial optic nerve transection; Curc—Curcumin; Resv—Resveratrol. RGC density was significantly higher in control group vs treatment. Significant improvement in RGC density in curcumin only and combined treatment groups compared to pONT only group. Data expressed as mean with SEM. Data analysed using One-Way ANOVA and post hoc Tukey analysis, *p<0.05**p<0.01.

FIG. 12A shows topical administration of Curcumin micelle (CN) eye drops (twice daily for three weeks) in an ocular hypertensive model (Morrison's model) showed significant neuroprotective effect versus OHT only controls. Unformulated Curcumin (FC) controls had no significant protective effect on RGC populations in this model. This model is often used as a model of retinal ganglion cell degeneration in glaucoma but the mechanisms of neurodegeneration are increasingly recognised to share strong similarities with other disorders of the CNS including Alzheimer's, PD etc. RGC density was determined histologically using Brn3a assessment as described in Davis et al. 2016 FIG. 12B shows that curcumin treatment has no effect on IOP suggesting the neuroprotective effects were IOP independent.

FIG. 13. Spectroscopic determination of curcumin content of nanocarrier formulations. [A] The keto- and enol-forms of curcumin (1,7-bis-(4-hydroxy-3-methoxyphenyl)-1,6-heptadiene-3,5-dione). [B] Suspensions of 4.5 mg/mL of curcumin in (left) PBS, (centre) PBS after 0.2 μm filtration to remove insoluble material and (right) in TPGS/Pluronic F127 nanoparticles after 0.2 μm filtration to remove insoluble material. [C] On dissolution in DMSO, 22 μM curcumin possesses an absorption peak of 435 nm. [D] Determination of the molar extinction coefficient of curcumin in a DMSO solvent (58,547 L·mol⁻¹·cm⁻¹) and demonstration that this accelerated oxidative degradation of curcumin at low pH⁶¹ results in a reduction in this molar extinction coefficient (2133 L·mol⁻¹·cm⁻¹ after 72 h incubation in 1M sodium hydroxide solution) suggesting that spectroscopic assessment can be used to monitor both the encapsulation of curcumin and its degradation. [E] Dissolution of 5 mg/mL curcumin in 1M sodium hydroxide solution (right) resulting in a rapid colour change compared to curcumin loaded nanocarriers (left). [F] Standard curve of known concentration of curcumin measured by HPLC.

FIG. 14. Characterization of curcumin loaded nanoparticles and stability assessment over time. [A] Transmission Electron Micrograph of curcumin loaded nanoparticles (CNs) negatively stained with 1% Uranyl acetate. Scale bar=50 nm. Dynamic light scattering revealed a homogeneous particle population which did not significantly change on storage at [B] 25° C. or [C] on lyophilization and storage at 25° C. and resuspension after 9 weeks. [D] Photograph of 1 mL lyophilized CN in 10 mM HEPES, 50 mg/mL trehalose buffer showing good cake structure. Stability studies illustrating the change in encapsulation efficiency over time when CN were stored at [E] 25° C. (solution) versus lyophilized and rehydrated. The average particle size [F] and dispersity index [G] was recorded in each case. Mean±95% CI.

FIG. 15. X-ray diffraction and FT-IR characterization of curcumin loaded nanocarrier formulations. [A] X-ray diffraction pattern of naïve curcumin (blue), empty nanocarriers (black) and curcumin nanocarriers (red). [B] FTIR analysis of (1) trehalose, (2) curcumin loaded nanoparticles, (3) free curcumin and (4) empty nanoparticles.

FIG. 16. In vitro release of curcumin. In vitro release of 4.5 mg/mL curcumin from [A] 95% ethanol solution or [B] curcumin-loaded nanocarriers in PBS at 37° C. (mean±SE, n=3). Owing to the poor solubility of curcumin in aqueous buffers, the release of curcumin from ethanoic solutions was limited by the formation of a visible precipitate from the 0.5 h time point. No such aggregation was observed in experiments using nanocarrier curcumin.

FIG. 17. Curcumin nanoparticle treatment is neuroprotective against the hypoxia mimetic cobalt chloride in immortalized retinal cells. Using an alamarBlue cell viability assay, Co-incubation of R28 cells with varying concentration of CNs significantly protected cells against [A, B] glutamate or [C, D] cobalt chloride induced insult (one-way ANOVA with Tukey post-test, ***p<0.001). Empty nanoparticles containing TPGS were found to be neuroprotective against glutamate induced toxicity [B] but not cobalt chloride [D] induced toxicity.

FIG. 18. Topical curcumin nanoparticles protect RGC soma in vivo against OHT induced cell loss. [A] Schematic of in-vivo experimental design. OHT rats were randomised to no treatment or once-daily curcumin nanoparticles (CN) or free Curcumin (FC) eye-drops, beginning two days prior to elevated IOP induction. Three-weeks after surgery, animals were sacrificed and retinas flat mounted before labelling with Brn3a. RGC populations were counted as previously described. Representative retinal images of comparable Brn3a labelled areas of superior retina are shown in [B] Naïve control, [C] OHT untreated and [D] OHT+CN animals. [E] All OHT animals had significantly raised TOP (mean±SE) versus baseline until 21 days after surgery (Student T-test versus contralateral eyes **p<0.01). There was no significant difference in TOP between OHT treatment groups at any time point suggesting any neuroprotective activity observed was TOP independent. [F] Elevated TOP in OHT only eyes was associated with a significant reduction in RGC density (˜23%) in agreement with previous studies; CN but not FC treatment, significantly reduced RGC loss (Kruskal-Wallis test with Dunns post test, **p<0.01).

FIG. 19. Topical curcumin nanoparticles protect RGC soma against optic nerve injury. Representative Brn3a labelled superior retinal sections taken from similar areas in [A] Naïve retina [B] pONT retina and [C] pONT model retina after daily topical CN treatment for 21 days. [D] Whole retinal RGC density measurements indicate that while pONT caused a significant reduction in RGC density, this was reduced by daily administration of CN (one-way ANOVA with Tukey post hoc test, **p<0.01 and ***p<0.0001). [E] Further segmentation of the each retina into superior and inferior quadrants using the method described previously (Davis et al. 2016 demonstrates that topical CN prevent some RGC density loss in both the superior and inferior retina (two-way ANOVA with Tukey post hoc tests ***p<0.001).

FIG. 20 shows the results of cell toxicity tests with the acylhomoserine derivative 3-HO—C₁₂ HSL, chosen due to its higher resistance to hydrolysis than 3-oxo-C12 HSL [Davis et al. 2011]. This molecule was found to be better tolerated than 3-oxo-C₁₂ HSL [A & B] and the common eyedrop additive and permeability enhancer Benzalkonium chloride (BAK) [C] in an immortalized human corneal epithelial cell line (HCE-S).

FIG. 21 shows [A] Negatively stained TEM micrograph of TPGS micelles loaded with 3-OH—C₁₂ HSL after filtration. 3-OH—C₁₂ HSL loaded micelles were prepared containing 1 mg/mL 3-OH—C₁₂ HSL with TPGS only [A & D], 5 kDa Chitosan with TPGS [B] and DSPE-PEG/Cholesterol micelles [D]. Owing to the low water solubility of 3-OH—C₁₂ HSL, unencapsulated material was removed by 0.22 μm filtration. 3-OH—C₁₂ HSL encapsulation was assessed by the ability of this molecule to reversibly disrupt HCE-S tight junctions after 2 h exposure to 300 μM concentrations of micelles containing up to 300 μM 3-OH—C12 HSL. Micelles must contain 3-OH—C12 HSL as vehicle only was unable to induce reversible reduction in HCE-S transepithelial resistance (TER, expressed as percentage of control, where control are micelles without the HSL).

FIG. 22 demonstrates that an acylhomoserine lactone (3-OH—C₁₂ HSL, abbreviated in this Figure as QS) mediates reversible disruption of Tight junctions and thus can be used for delivery of therapeutic molecules across biological barriers. Using an in vitro HCE-S transwell barrier model as described in Davis et al. (2014), transcytosis of [Ai-Aiii] 40 kDa FITC Dextran and [Bi-Biii] 149 kDa Avastin (the antibody bevacizumab) was significantly enhanced after pre-treatment of HCE-S for 2 h with 300 μM 3-OH—C₁₂ HSL. Controls were equivalent formulations without the AHSL (black circles in the graphs). This demonstrates the drug delivery enhancement potential of AHSLs.

FIG. 23 shows that co-administration of 3-OH—C12 HSL-containing micelles with a model drug molecule (40 kDa Anx-FITC (annexin V conjugated with the dye fluorescein isothiocyanate) was sufficient to promote the delivery of this agent across a HCE-S transwell model of biological barriers.

FIG. 24 shows that AHL containing formulations (3-OH—C12 HSL) was well tolerated using the in vitro HETCAM test [A] and in vivo Drazie test [B] at concentrations of 0.3 mM (F1) and 0.6 mM (F2) versus vehicle controls (TPGS micelles). These results are in stark contrast to other permeability enhancers such as Benzalkonium chloride (BAK) which are known to induce potent ocular irritation in both rabbits and humans.

FIG. 25 shows results of a stability assessment of a formulation of 4.5 mg/ml curcumin encapsulated in micelles formed from 25 mg/ml TPGS and 150 mg/ml solutol, measuring encapsulation efficiency (A), particle size (B), and polydispersity index (PDI) (C) over time.

FIG. 26 shows results of a stability assessment of encapsulation efficiency (A), particle size (B), and polydispersity index (PDI) (C) of the formulation of FIG. 25 after freeze-drying and subsequent rehydration.

FIG. 27 shows that intranasal administration of the curcumin micelles (B, C) decreased the DARC count in the retina of a 3xTg-AD mouse model when compared to vehicle alone (A).

FIG. 28 shows that intranasal administration of the curcumin micelles (B, C) protected against retinal ganglion cell (RGC) loss in the 3xTg-AD mouse model when compared to vehicle alone (A).

FIG. 29 shows that intranasal administration of the curcumin micelles (B, C) reduced amyloidβ deposition in the hippocampus of 3xTg-AD mice when compared to vehicle alone (A).

FIG. 30 shows results of a stability assessment of a formulation of 15 mg/ml resveratrol encapsulated in micelles formed from 25 mg/ml TPGS and 150 mg/ml solutol, measuring encapsulation efficiency (A), particle size (B), and polydispersity index (PDI) (C) over time.

FIG. 31 shows results of a stability assessment of encapsulation efficiency (A), particle size (B), and polydispersity index (PDI) (C) of the formulation of FIG. 30 after freeze-drying and subsequent rehydration.

FIG. 32: resveratrol nanoparticle treatment is neuroprotective against glutamate excitotoxicity in immortalized retinal cells. Using an alamarBlue cell viability assay, Co-incubation of R28 cells with RNs significantly protected cells against glutamate induced insult (A) (one-way ANOVA with Tukey post-test, ***p<0.001). Glutamate toxicity (B). Empty nanoparticles containing TPGS were found to be neuroprotective against glutamate induced toxicity (A, C). Protective effect of RNs (A, D).

FIG. 33: resveratrol nanoparticle treatment is not neuroprotective against cobalt chloride induced hypoxia in immortalized retinal cells (A). Control (B), empty nanoparticles (C) and RNs (D).

FIG. 34 shows intranasal administration of the resveratrol micelles decreased the DARC count in the retina of a 3xTg-AD mouse model when compared to vehicle alone.

FIG. 35 shows that intranasal administration of the resveratrol micelles (B, C) reduced amyloidβ deposition in the hippocampus of 3xTg-AD mice when compared to vehicle alone (A).

### EXAMPLES

### Example 1

Micelles comprising TPGS and Lutrol F127 with up to 5 mg/mL of curcumin or Resveratrol have been prepared and tested for stability. Micelles have also been prepared using PEG-Cholesterol with Lutrol F127.

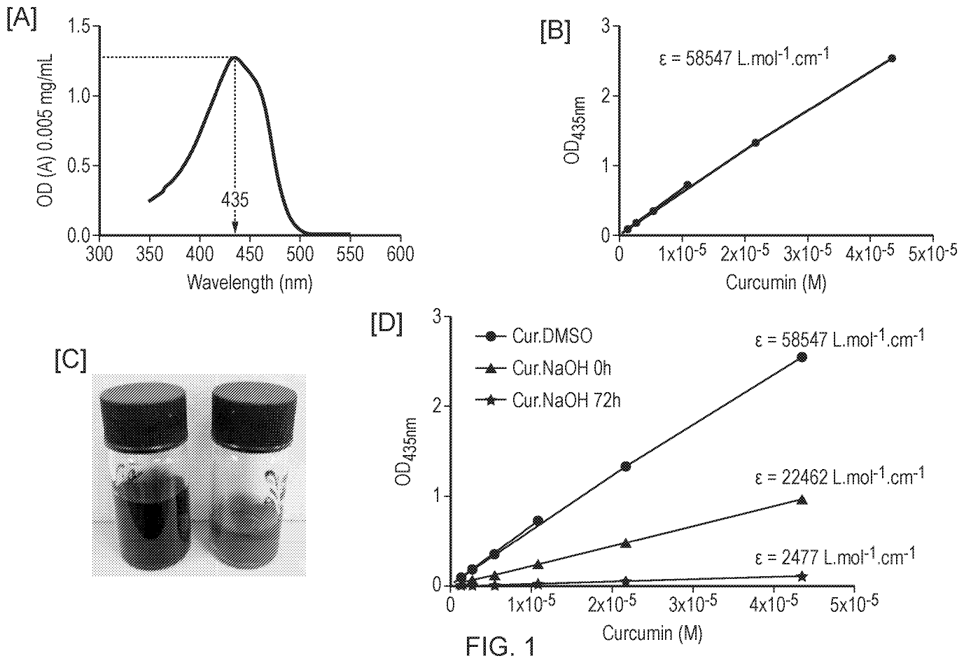

As shown in FIG. 1, the concentration of curcumin in formulations was determined spectroscopically. This enabled us to determine not only the concentration of curcumin in each formulation but also the amount of active drug as hydrolysed curcumin (which is therapeutically useless) has practically no absorbance at 435 nm relative to the in-tact drug. (Forced curcumin degradation achieved on treatment with sodium hydroxide (FIG. 1D, red line)).

### Method of Manufacture

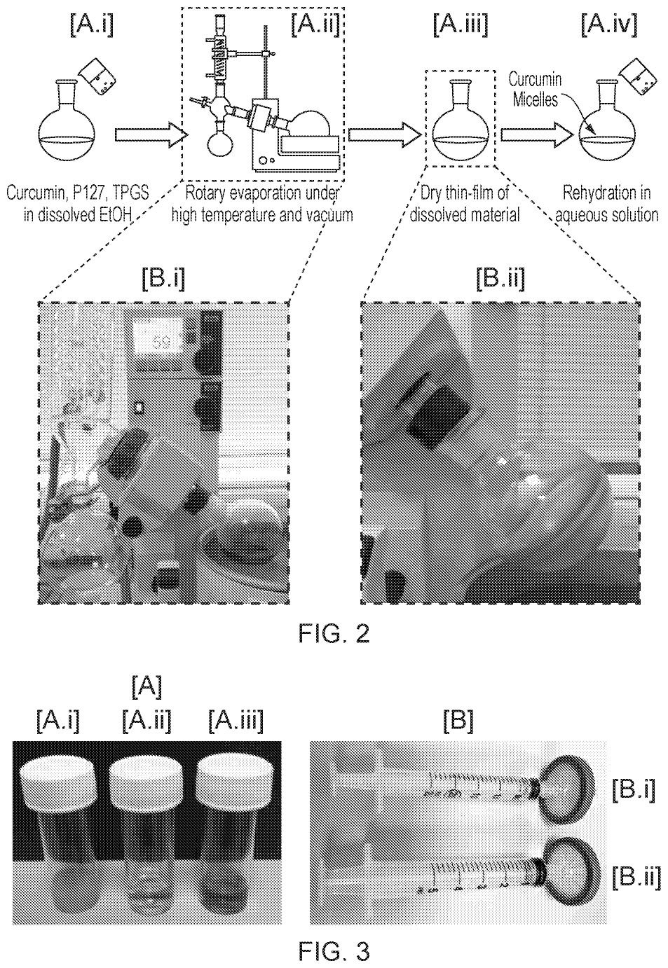

Curcumin micelles were formed by a thin-film rehydration method (see FIG. 2). Curcumin, P127 and TPGS were first dissolved in absolute ethanol (Fisher Scientific, UK) via ultrasonication to form stock solutions. These were added in desired molar ratios (Table 1) to a round-bottomed flask and vortexed to mix. Ethanol solvent was evaporated using a vacuum-assisted rotary evaporator (Rotavapor R-210/Vacuum Controller V-850, Buchi, Switzerland) under 50 mbar vacuum and at the desired temperature (Table 1) until a dry thin-film of dissolved material remained. Dry films were resuspended in various aqueous buffer solutions at high temperature on the rotary evaporator (1 bar). The resulting micelles were separated from unencapsulated (insoluble) curcumin by filtration through a 0.22 μm filter (33 mm Millex filter, Merck Millipore, USA). The resulting micelle formulation was then characterised through determination of curcumin encapsulation efficiency, particle size and stability over time. Rehydration buffers included; distilled water, phosphate buffer solution (PBS) and Tris (20 mM) Trehalose (50 mg/mL) containing buffers. For topical instillation a neutral pH is preferred hence drug stability was investigated whilst in PBS buffer.

TABLE 1
Individual formulation protocol and composition details

| | Rotavap: | | | | | | | | |
|---|---|---|---|---|---|---|---|---|---|
| | Temp (° C.), | Resuspension | | Composition | | | | | |
| | vacuum (mbar) | Temp (° C.), | | EtOH | Molarity (mM) | | | Veh:Cur | P127:TPGS |
| # | duration (h) | duration (h) | Solvent | (ml) | TPGS | Cur | P127 | ratio | ratio |
| F1 | 65, 50, 2 | 50, 0.5 | 1x PBS | 80 | 22.55 | 12.22 | 0.00 | 2.50 | 0.00 |
| F2 | 65, 50, 2 | 50, 0.5 | 1x PBS | x | 22.55 | 12.22 | 3.97 | 2.17 | 0.18 |
| F3 | 50, 50, 18 | 50, 0.5 | dH20 | 10 | 3.83 | 6.11 | 1.35 | 0.85 | 0.35 |
| F4 | 50, 50, 18 | 60, 0.5 | dH20 | 10 | 3.83 | 6.11 | 1.35 | 0.85 | 0.35 |
| F5 | 50, 50, 18 | 60, 0.5 | Trehalose | 10 | 3.83 | 6.11 | 1.35 | 0.85 | 0.35 |
| | | | 50 mg/mL | | | | | | |
| F6 | 65, 50, 2 | 50, 1 | 1x PBS | x | 5.00 | 6.11 | 10.32 | 2.51 | 2.06 |
| F7 | 50, 50, 18 | 60, 0.5 | dH20 | 20 | 7.67 | 12.22 | 2.70 | 0.85 | 0.35 |
| F8 | 50, 50, 18 | 60, 0.5 | Trehalose | 20 | 7.67 | 12.22 | 2.70 | 0.85 | 0.35 |
| | | | 50 mg/mL | | | | | | |
| F9 | 50, 50, 18 | 60, 0.5 | Trehalose | 20 | 7.67 | 12.22 | 2.70 | 0.85 | 0.35 |
| | | | 50 mg/mL | | | | | | |
| F10 | 50, 50, 18 | 60, 0.5 | Trehalose | 20 | 7.67 | 12.22 | 2.70 | 0.85 | 0.35 |
| | | | 50 mg/mL | | | | | | |
| F11 | 65, 50, 2 | 50, 0.5 | dH20 | X | 11.27 | 6.11 | 3.97 | 2.50 | 0.35 |
| F12 | 65, 50, 2 | 50, 0.5 | 1x PBS | x | 11.27 | 6.11 | 3.97 | 2.50 | 0.35 |
| F13 | 65, 50, 2 | 50, 0.5 | 1x PBS | x | 11.27 | 6.11 | 3.97 | 2.50 | 0.35 |
| F14 | 65, 50, 2 | 50, 0.5 | 1x PBS | x | 11.27 | 6.11 | 3.97 | 2.50 | 0.35 |
| F15 | 60, 50, 4 | 50, 0.5 | 1x PBS | x | 22.55 | 12.22 | 7.94 | 2.50 | 0.35 |
| F16 | 65, 50, 2 | 50, 0.5 | 1x PBS | x | 22.55 | 12.22 | 7.94 | 2.50 | 0.35 |
| F17 | 65, 50, 2 | 50, 0.5 | 1x PBS | x | 22.55 | 12.22 | 7.94 | 2.50 | 0.35 |
| F18 | 65, 50, 2 | 50, 0.5 | 1x PBS | x | 22.55 | 12.22 | 7.94 | 2.50 | 0.35 |
| F19 | 65, 50, 2 | 50, 0.5 | 1x PBS | x | 22.55 | 12.22 | 7.94 | 2.50 | 0.35 |
| F20 | 65, 50, 2 | 50, 0.5 | 1x PBS | x | 22.55 | 12.22 | 7.94 | 2.50 | 0.35 |
| F21 | 65, 50, 2 | 50, 0.5 | 1x PBS | x | 22.55 | 12.22 | 7.94 | 2.50 | 0.35 |
| F22 | 65, 50, 2 | 50, 0.5 | 1x PBS | x | 22.55 | 12.22 | 7.94 | 2.50 | 0.35 |
| F23 | 65, 50, 2 | 50, 0.5 | 1x PBS | x | 22.55 | 12.22 | 7.94 | 2.50 | 0.35 |
| F24 | 65, 50, 2 | 50, 1 | 1x PBS | x | 90.20 | 48.86 | 31.75 | 2.50 | 0.35 |
| F25 | 65, 50, 2 | 50, 0.5 | Tris: | x | 22.55 | 12.22 | 7.94 | 2.50 | 0.35 |
| | | | Trehalose | | | | | | |
| | | | 50 mg/mL | | | | | | |
| F26 | 65, 50, 2 | 50, 0.5 | Trehalose | x | 22.55 | 12.22 | 7.94 | 2.50 | 0.35 |
| F27 | 65, 50, 2 | 50, 0.5 | 1x PBS | x | 22.55 | 0.00 | 7.94 | n/a | 0.35 |

Descriptions of formulations produced in this study. dH20 (distilled water); PBS (phosphate-buffered); EtOH (ethanol); Cur (curcumin); TPGS (D-α-tocopheryl polyethylene glycol 1000 succinate); P127 (Pluronic F-127)

Unencapsulated curcumin was removed by filtration as shown in FIG. 3.

TABLE 2
Characterisation and Stability results (Curcumin micelles)

| | | | | [Curcumin] | EE | Z-average | | Stability |
|---|---|---|---|---|---|---|---|---|
| Type | Formulation # | n | Solvent | (mg/mL) | (%) | (nm) | PDI | (days) |
| D | F11 | 1 | dH₂0 | 1.68 | 75 | 16.47 | 0.169 | 48 |
| E | F12, F13, | 3 | PBS | 1.99 ± 0.08 | 88 ± 4 | 19.45 ± 0.62 | 0.094 ± 0.04 | 37 ± 8 |
| | F14 | | | (n = 3) | (n = 3) | (n = 3) | (n = 3) | (n = 2) |
| F | F15, F16, | 9 | PBS | 4.31 ± 0.07 | 96 ± 2 | 21.08 ± 0.79 | 0.098 ± 0.027 | 40 ± 6 |
| | F17, F18, | | | (n = 7) | (n = 7) | (n = 9) | (n = 9) | (n = 2) |
| | F19, F20, | | | | | | | |
| | F21, F22, F23 | | | | | | | |

TABLE 3
Freeze-drying: A three-step process

| | | | |
|---|---|---|---|
| Process | Temperature | Pressure | Duration |

| | | | | | | |
|---|---|---|---|---|---|---|
| Freezing | −60° | C. | 1 | bar | 2 | h |
| 1° Drying | −42° | C. | 200 | mTorr | 24 | h |
| 2° Drying | 20° | C. | 200 | mTorr | 12 | h |

Freeze-drying protocol involved three stages. 1° (Primary), 2° (Secondary); h (hours).

TABLE 4
Freeze-drying and Rehydration Long-term Stability (Preliminary results in PBS)

| | | | Freeze-drying | | | 1 week Rehydration | | |
|---|---|---|---|---|---|---|---|---|
| Time | | | EE | Z-Average | | EE | Z-Average | |
| (weeks) | Solvent | N | (%) | (nm) | PDI | (%) | (nm) | PDI |
| 6 | dH20 | 1 | 1 | 18.88 | 0.048 | | | |
| 7 | dH20 | 1 | 89 | 18.17 | 0.083 | | | |
| 7 | PBS | 2 | 96 ± 9 | 19.61 ± 0.02 | 0.035 ± 0.003 | 90 | 21.2 | 0.206 |
| 8 | PBS | 2 | 93 ± 7 | 20.12 ± 0.05 | 0.038 ± 0.007 | 71 ± 0 | 68.84 ± 47.46 | 0.179 ± 0.018 |

Cryoprotectant later replaced with 10 mg/mL to 100 mg/mL Trehalose (with 10 mM HEPES, pH 7.4) which showed better stability as shown in following formulations.

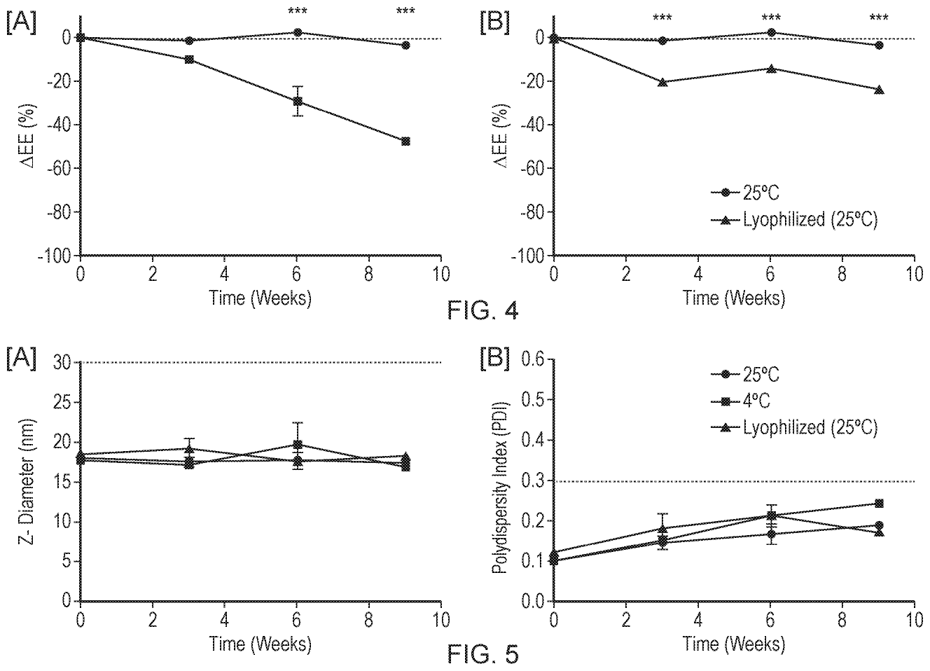

Curcumin micelle stability was good at room temperature and after freeze-drying in the presence of a trehalose cryoprotectant (10 mg/mL to 100 mg/mL trehalose). Micelles were prepared at both pH 4.5 and pH 7.4. See FIGS. 4 and 5.

Resveratrol micelle formulation (later co-administered with curcumin micelles in vivo) showed good stability using the same micelle formulation (see Table 5).

TABLE 5
Configurations of initial formulations produced in study: #—Formulation
configuration code; PBS—Phosphate Buffered Saline; Trehalose—Hepes-
Trehalose (50 mg/mL); TPGS—D-α-tocopheryl polyethylene glycol 100
succinate; P127—Pluronic F-127; Res—Resveratrol

| | Rotary Evaporator | | | | | | | |
|---|---|---|---|---|---|---|---|---|
| | Settings | | | | | | | Starting |
| | Temp (° C.), | | | Molarity of excipients | | | Dilution | conc., of |
| | Vacuum (mBar), | Number of | | (M) | | | factor when | product |
| | Duration (hr) | formulations | Buffer | TPGS | P127 | Res | characterising | (mg/ml) |
| A | 65, 50, 2 | 1 | PBS | 22.36 | 8 | 19.72 | 1:100 | 5 |
| B | 65, 50, 2 | 2 | PBS | 22.36 | 8 | 19.72 | 1:250 | 5 |
| C | 65, 50, 2 | 1 | Trehalose | 22.36 | 8 | 15.77 | 1:500 | 4 |
| D | 65, 50, 2 | 1 | Trehalose | 22.36 | 8 | 19.72 | 1:500 | 5 |
| E | 65, 50, 2 | 1 | Trehalose | 22.36 | 8 | 23.66 | 1:500 | 6 |

TABLE 6
Amount of encapsulated resveratrol produced by
different starting concentrations of the drug

| Starting resveratrol | Encapsulation | Resveratrol |
|---|---|---|
| concentration (mg/mL) | efficiency (%) | encapsulated (mg/mL) |
| 4 | 78.37 | 3.13 |
| 5 | 94.27 | 4.71 |
| 6 | 83.02 | 4.98 |

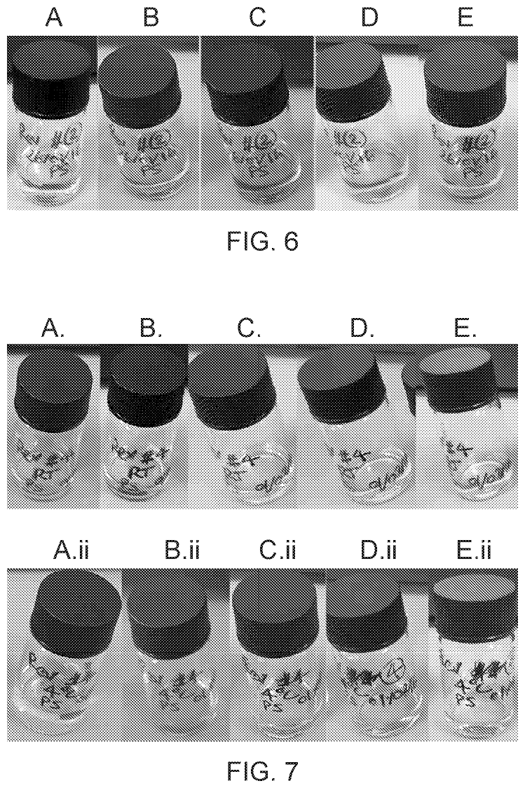

Preferred buffer composition is with Trehalose (10 mg/mL to 100 mg/mL) as this antioxidant prevents degradation of the product on storage over time versus PBS which causes a progressive discolouration of the product (see FIGS. 6 and 7).

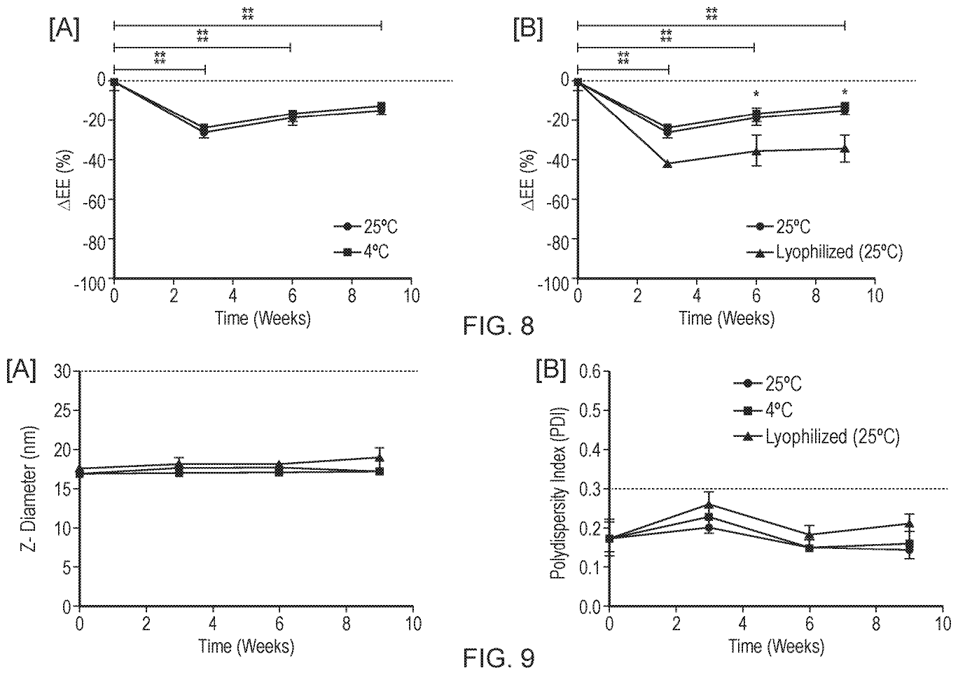

Samples showed good stability for up to 10 weeks (See FIGS. 8 and 9).

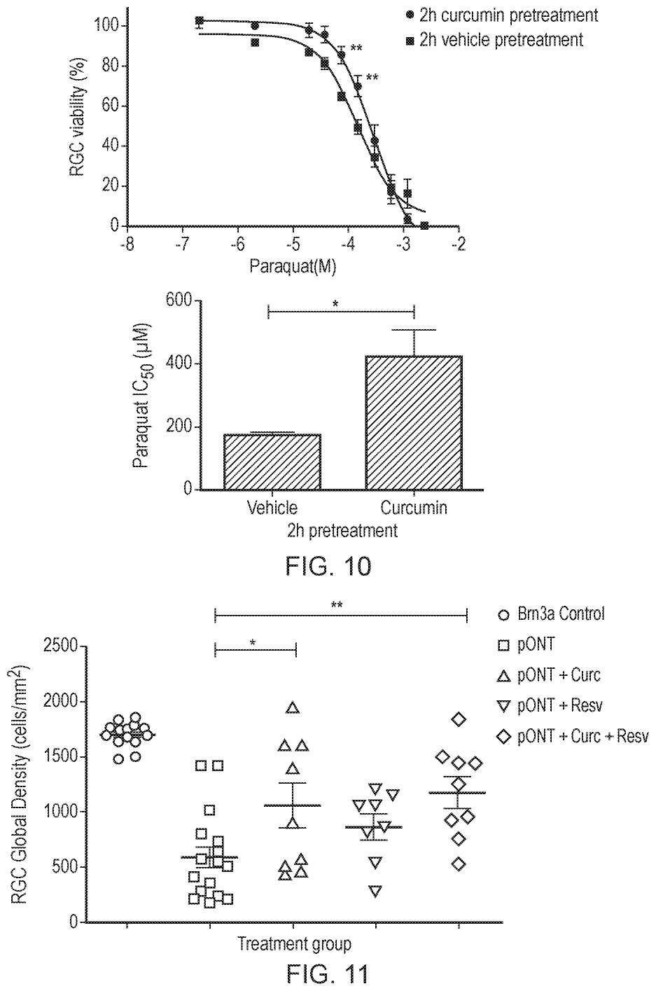

The curcumin micelle formulation was found to be significant neuroprotective in vitro (see FIG. 10).

Topical curcumin micelle eye drops (twice a day for three weeks) and curcumin/resveratrol micelle co-therapy (two drops of each per day) were found to be significantly neuroprotective in a rat model of optic neuropathy (partial optic nerve transection) (FIG. 11). This neurodegenerative model is predominantly used to assess the efficacy of therapy against retinal disorders but owing to the similarities in generative processes between the retina and brain can be used to inform therapeutic potential in other CNS disorders. These results suggest a synergism from PPAR-g activity modulator curcumin and the antioxidant resveratrol using our micelle formulations.

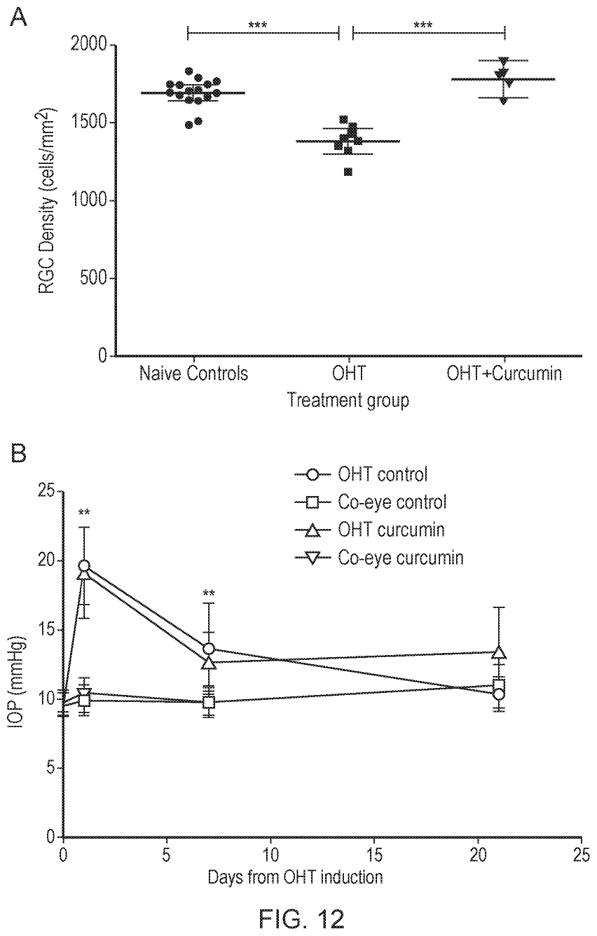

Curcumin treatment had no effect on TOP suggesting the neuroprotective effects were TOP independent (see FIG. 12).

### Example 2

### Methods and Materials

### Preparation of Curcumin Loaded Nanocarriers

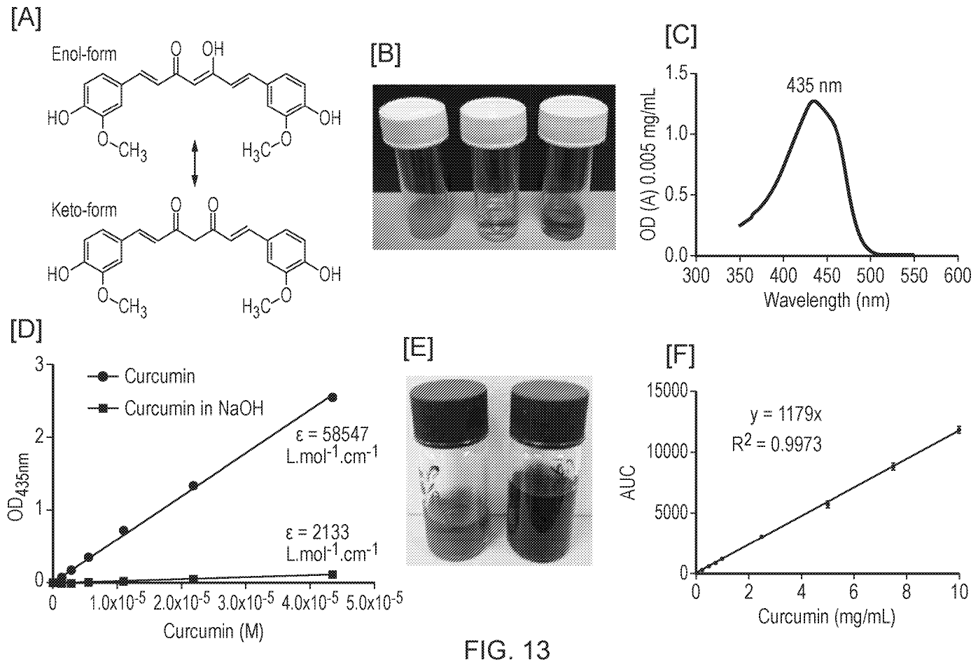

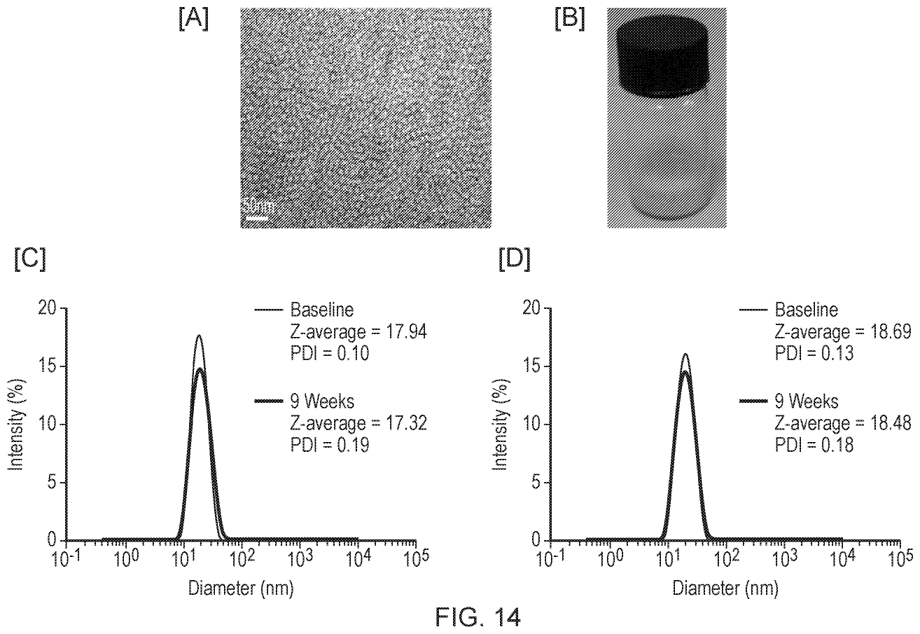

Curcumin, D-α-tocopherol polyethene glycol 1000 succinate (TPGS) and Pluronic F127 were obtained at the highest available purity from Sigma-Aldrich (Kent, UK). Curcumin-loaded nanocarriers (CN) were prepared using an adaptation of the thin-film hydration technique described previously (Davis et al. 2014). Curcumin, TPGS, and Pluronic F127 were dissolved in ethanol to a concentration of 5 mg/mL, 10 mg/mL and 20 mg/mL respectively; with 10 min of gentle heating and bath ultrasonication to clarity. Solutions were aliquoted in the desired molar ratio (22.55 mM, 12.22 mM 7.94 mM of TPGS, curcumin, and Pluronic F127 respectively) into a round bottom flask, mixing well. The solvent was removed by rotary evaporation (50 mBar, 65° C., 2 h) using a Rotavapor R210 with a V850 Vacuum controller (Buchi, Switzerland) while protecting from light. After this time, the thin-film was rehydrated (50° C., 0.5 h) in the desired buffer (distilled water, phosphate buffered saline (pH 7.4) or HEPES trehalose buffer (10 mM HEPES, 50 mg/mL trehalose, pH 7.4). Unencapsulated curcumin was then removed from the formulation by 0.22 μm filtration (33 mm Millex filter, Merck Millipore, USA) as shown in FIG. 13B. Free-curcumin (FC) was prepared according to the same protocol as described above, without the addition of TPGS or Pluronic F127.

### Lyophilisation of Curcumin Loaded Nanocarriers

Lyophilisation of CN formulations in HEPES trehalose buffer was achieved by equilibrating 1 mL aliquots of nanocarriers in 7 mL screw neck squat form glass vials (CamLab, Cambridge UK) at 25° C. before freezing at −60° C. for 2 h at 760 Torr. Primary drying of samples was completed at −38° C. at 200 mTorr for 24 h, followed by a secondary drying phase at 25° C. and 200 mTorr for 2 h. Samples were capped immediately after cessation of secondary drying before storing at 25° C. while protecting from light until required. For stability assessment, samples were rehydrated for 30 minutes by addition of 1 mL of 0.22 μm filtered distilled water with gentle mixing.

The moisture content of formulations was determined using thermogravimetric analysis (TGA). Freeze dry samples were placed in an aluminium pan and analysed by a Discovery TGA (TA instruments, USA). Samples were purged with a flow rate of 25 mL/min nitrogen gas and heated from 30 to 200° C. with 10° C./min rate. The percent mass loss was calculated by TA Instruments Trios software at 120° C. for water content. Three freeze dry formulations were measured three times for each sample.

### Curcumin Loading Efficiency

The loading efficiency of CNs was determined spectroscopically and results confirmed using HPLC. Spectroscopic determination of curcumin loading was achieved by diluting in DMSO 1:500 at 435 nm normalised to empty nanocarriers. The concentration of curcumin in each formulation was then determined using the molar extinction coefficient of curcumin (FIG. 13) determined by constructing a standard curve measuring the absorbance of known curcumin concentrations. The encapsulation efficiency of each formulation was calculated using equation 1;

$$EE \% = 100 \cdot ( \frac{{[ C ]}_{E}}{{[ C ]}_{S}} ) \qquad [ 1 ]$$

where [C]s is the concentration of curcumin originally added to the formulation (typically 4.5 mg/mL) and [C]E is the concentration of curcumin detected spectroscopically within the nanocarriers after 0.22 μm filtration to remove unencapsulated material. Results were confirmed using an adaptation of an established HPLC technique (Guddadarangavvanahally et al.). Briefly, curcumin containing samples were diluted in methanol before 20 μL volumes were injected at 25° C. onto a Phenomenex® Synergi (4 μm Polar—RP 80 μL with size of 250×4.60 mm) column with an Acetonitrile: 0.1% trifluoroacetic acid 50:50 solvent system at a flow rate of 1 mL/min connected to a Agilent Technology 1260 Infinity HPLC system. Absorbance was recorded at 420 nm and the area under the curcumin elution curve compared to a standard curve of known curcumin concentrations.

### Dynamic Light Scattering

Particle size was determined using a Malvern Zetasizer. Measurements of particle diameter and polydispersity index were recorded from a minimum of three formulations for each experimental condition or time point after manufacture. Nanocarriers were diluted 1 in 10 in the appropriate buffer prior to recording.

### Transmission Electron Microscopy

Nanocarrier suspensions were processed using carbon grids to absorb particles from suspension before staining with 1% uranyl acetate for 1 min and drying. Specimens were observed using a Joel-1010 Transition Electron Microscope operated at 100 kV with images acquired using a Gatan Orius digital camera.

### X-Ray Diffraction and FT-IR

X-ray diffraction graphs of drug alone, empty nanoparticles or CN were prepared from X-ray diffractometer (Rigaku MiniFlex 600) and the 2-thea angle was set from 5° to 65° with an angular increment of 0.05°/second. The measurements were performed at a voltage of 40 kV and 15 mA. The FT-IR spectrum of free curcumin, empty nanoparticles and CN were recorded using a PerkinElmer Spectrum 100 FT-IR spectrometer at 4 cm-1 resolution, with 4 scans between 4000 cm-1 and 650 cm-1.

### Curcumin Release Assay

In vitro curcumin release was assessed using an adaptation of a previously described protocol (Wang et al. 2012). Briefly, free curcumin (dissolved in 95% ethanol) or CNs containing 4.5 mg/mL of curcumin was loaded into a 1 mL Spectra-Por Float-A-Lyzer dialysis cassette (Sigma-Aldrich) with 3.5-5 kDa molecular weight cut-off. Samples were dialysed against 200 mL of PBS containing 10% Tween-80 to act as a sink for released curcumin maintained at 37° C. with stirring at 50 rpm. At the specified time points, samples were removed from the mixture and replaced with fresh buffer. The concentration of curcumin was determined as described above. Results from three experimental replicates were fit to a single phase association (equation 2);

Y=Y0+(Plateau−Y0)*(1−exp(−K*x)) (2)

Where Y₀=zero, Plateau is the maximal release and K is the rate of curcumin release (h⁻¹) from which half-life (t_{1/2}) was calculated (t_{1/2}=ln 2/K).

### Cell Culture

R28 cell line (Kerafast, Boston, Mass.) were cultured in Dulbecco's modified Eagle's medium (DMEM; Invitrogen, Paisley, UK) supplemented with 5% foetal bovine serum (Invitrogen, UK), 100 U/ml penicillin, 100 m/ml of streptomycin and 0.292 mg/mL glutamine (Gibco, UK), 7.5% sterile dH20 and 1.5 mM KCl (Sigma-Aldrich, UK). The medium was changed every other day and cultures were passaged at 90% confluence.

### Cell Viability Assessment

R28 cells were plated at 4,000 cells/well in 96-well plates for 24 h before treatment with varying concentrations of curcumin (0 to 20 μM) or an equivalent concentration of TPGS/Pluronic F127 only nanocarriers (vehicle control) in conjunction with varying concentrations of cobalt chloride or glutamate insults for a further 24 h. Cell viability was assessed in each case using the Alamarblue (Invitrogen, UK) assay according to manufacturer's instructions. Briefly, the Alamarblue solution was added to each well-plate and incubated for 4 hours before recording the fluorescence using a Safire plate reader excitation of 530 nm and emission of 590 nm 91.

### Animals

All animal experiments were performed with procedures approved by the U.K. Home Office and in accordance with the ARVO Statement for the Use of Animals in Ophthalmic and Vision Research. For in vivo assessment of experiments: in total 48 Adult male Dark Agouti (DA) rats (Harlan Laboratories, UK) weighing 150 to 200 g were housed in an air-conditioned, 21° C. environment with a 12 h light-dark cycle (140-260 lux), where food and water were available ad libitum. 13 animals served as naïve controls which were not subject to further interventions before immunohistochemistry.

### Ocular Hypertension (OHT) Model

Ocular hypertension was surgically induced in the left eye of 18 DA rats (5 OHT only, 5 OHT+CN, 8 OHT+FC) as described previously (Morrison et al. 1997). Procedures were conducted under general anaesthesia using a mixture of 37.5% Ketamine (Pfizer Animal Health, Exton, Pa.), 25% Dormitol (Pfizer Animal Health, Exton, Pa.) and 37.5% sterile water, at 2 mL/kg administered intraperitoneally. Briefly, 50 μL of hypertonic saline solution (1.8 M) was injected into the two episcleral veins using a syringe pump (50 μL/min; UMP2; World Precision Instruments, Sarasota, Fla., USA). A propylene ring with a 1 mm gap cut from the circumference was placed around the equator to prevent injected saline outflow from other aqueous veins. The IOP from both eyes of each rat was measured at regular intervals using a TonoLab tonometer (Tiolat Oy, Helsinki, Finland) under inhalational anaesthesia (0.4% isoflurane in oxygen). Daily administration of topical CNs was performed in 5 DA rats (two 35 μL drops/day 5 min apart at 10 am each day) starting two days prior to model induction and continuing until model termination (21 days post IOP elevation) with 5 rats serving as OHT only controls. An additional 8 rats received free-curcumin (FC) prepared using the same protocol as CN curcumin without the addition of TPGS or Pluronic F127. FC was administered to OHT animals using the same dosing regime as described for CN. Animals were sacrificed three weeks after unilateral IOP elevation and retinas flat-mounted prior to Brn3a immunohistochemistry.

### Partial Optic Nerve Transection (PONT) Model

Partial optic nerve transection was conducted in the left eye of 17 DA rats, using a previously described technique (Levokovitch-Verbin et al. 2003). Under general anaesthesia, an incision was made in the superior conjunctiva, and the ON sheath was exposed. A longitudinal slit was next formed in the dura mater to expose the ON and a 0.2-mm cut was made in the dorsal ON, 2 mm behind the eye using an ophthalmic scalpel with steel cutting guard. Damage to major ophthalmic blood vessels was avoided and verified at the completion of surgery by ophthalmoscopy. Daily administration of topical CNs was conducted in 9 DA rats after induction of the pONT model using the same treatment regimen as described previously with the remaining 8 serving as pONT only controls.

### Brn-3a Immunohistochemistry and Confocal Microscopy

Brn-3a labelling of RGCs in retinal whole-mounts was completed as described previously (Davis et al. 2016). Briefly, eyes were enucleated upon sacrifice and fixed in 4% paraformaldehyde at 4° C. overnight before dissecting retinal whole mounts. Whole mounts were stained for the RGC specific nuclear-localised transcription factor Brn3a using an anti-mouse mAb (1:500, Merck Millipore, Darmstadt, Germany) and examined under confocal microscopy (LSM 710, Carl Zeiss MicroImaging GmbH, Jena, Germany). Each retinal whole mount was imaged as a tiled z-stack at ×10 magnification which was used to generate a single plane maximum projection of the RGC layer in each retina for subsequent analysis. Each whole mount image was manually orientated so that the superior retina was towards the top of the image using in vivo cSLO imaging of retinal vasculature as a reference. Retinal image acquisition settings were kept constant for all retinas imaged, allowing comparison of Brn3a expression in each experimental group as previously described. 94 Automated quantification of Brn3a labelled RGCs in retinal whole-mounts was completed as described previously (Davis et al. 2016).

### Statistical Analysis

All data were analysed with the Student's t-test, ANOVA or with appropriate post hoc testing using GraphPad Prism 5 (GraphPad Software, Inc., La Jolla, Calif., USA) as appropriate. Data were presented as means±SE and p<0.05 was considered significant. Molecular structures were drawn using ACD/ChemSketch 2015 and all images were taken by the authors (BMD).

### Results and Discussion

### Spectroscopic Methods can be Used to Assess Curcumin Encapsulation Efficiency and Oxidation State

On dilution in dimethyl sulfoxide (DMSO) curcumin had an absorbance peak at 435 nm (FIG. 13C) and molar extinction coefficient (FIGS. 13D&E) of 58547 L·mol⁻¹·cm⁻¹, comparable to previously reported values. The absorbance of curcumin diluted in DMSO at 435 nm obeyed Beer-Lambert's law up to 42 μM and TPGS/Pluronic F127 nanocarriers in the absence of curcumin had no measurable absorbance at this wavelength. Spectroscopic assessment was used to determine the encapsulation efficiency (EE %) of curcumin-containing formulations after separation of unencapsulated material by 0.22 μm filtration. Spectroscopic measurements of EE % were confirmed using an established HPLC technique (FIG. 13F) with both techniques showed good agreement (4.31±0.18 mg/mL versus 4.32±0.33 mg/mL respectively).

Spectroscopic determination of curcumin concentration in nanocarriers can also be used to give indication of the extent of curcumin degradation. Curcumin undergoes keto-enol tautomerization (FIG. 13A), existing in the more stable keto form under acidic or neutral conditions and the more water soluble enol-form under alkaline conditions. In common with other molecules that undergo keto-enol tautomerization, the enol form of curcumin is more prone to hydrolytic degradation. Acceleration of curcumin degradation processes by dissolution in an alkaline buffer, gave rise to a dramatically reduced curcumin molar extinction coefficient at 435 nm compared to formulated curcumin (FIG. 13C-D, 2133 L·mol⁻¹·cm⁻¹ after 72 h incubation in the presence of 1 M sodium hydroxide solution). Furthermore, incubation of CNs in alkaline conditions induced a dramatic colour change from orange to brown (FIG. 13E). Spectroscopic assessment of curcumin concentration after dissolution in sodium hydroxide indicates that the curcumin molar extinction coefficient rapidly diminished, suggesting that this technique can not only be used to assess curcumin entrapment efficiency but also be used to monitor the extent of degradation of curcumin containing formulations.

### TPGS/Pluronic F127 Nanocarriers Enhance Curcumin Solubility and Stability

Initially, curcumin loaded nanocarriers were prepared by incorporation it into TPGS nanocarriers. TPGS was chosen due to the low critical micelle concentration of this excipient (0.02% w/w), the endogenous nature and antioxidant properties of the α-tocopherol component and P-glycoprotein antagonism, which enhances the barrier crossing ability of formulations containing this agent. TPGS is present in existing ophthalmic formulations and both curcumin and TPGS can be readily solubilized in ethanol, a solvent which is present at concentrations of 0.8% in commercially available eye drop formulations (i.e. Optrex ActiMist 2in 1 Eye Spray for Dry Irritated Eyes) so reducing risks associated with residual solvents from the manufacturing process. Furthermore, as the use of TPGS to enhance the bioavailability of orally administered drugs is well documented. This, in combination with recent interest in the use of Pluronic F127 food-research applications may suggest that the novel curcumin formulation described herein may also be suitable for oral administration.

Formulation of curcumin with TPGS micelles was found to produce nanocarriers with 16 nm diameter as determined by dynamic light scattering (data not shown). Unfortunately, these formulations rapidly aggregated at 25° C., resulting in the formation of sediment within hours of resuspension which may be indicative of Ostwald ripening processes. Stabilisation of curcumin loaded TPGS nanocarriers was achieved by the addition of the polymeric stabiliser Pluronic F127 (a triblock copolymer of polyoxyethylene and polyoxypropylene), which has previously been used to sterically stabilise nanocarriers against aggregation.

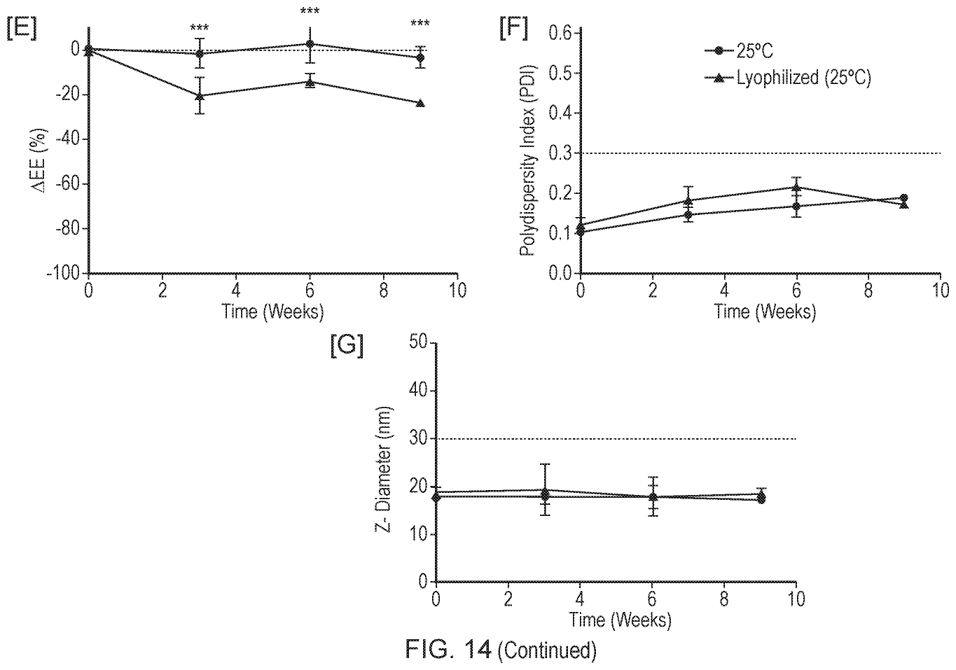

Curcumin-loaded nanocarriers (CN) were prepared according to the methods described, with encapsulation efficiency and average particle size determined (FIG. 14). On resuspension in PBS (pH 7.4) or HEPES trehalose buffer (pH 7.4), nanocarriers were found to encapsulate 96.0%±2.0% (4.32 mg/mL) and 94.2%±4.1% (4.31 mg/mL) of curcumin respectively. Transmission electron microscopy revealed that nanocarriers were typically 20 nm in diameter and of uniform size (FIG. 14A). These results were confirmed by dynamic light scattering (FIG. 14B-C) which identified a homogeneous particle dispersion with a z-average diameter between 16 and 20 nm suggestive of a micellar formulation.

The encapsulation efficiency and particle size of CN formulations were assessed over time after storage at 25° C. while protecting from light. The CN formulation was found to exhibit excellent stability for 9 weeks at 25° C., with no reduction in formulation EE % (FIG. 14E), significant change in particle diameter (FIG. 14F) or dispersity (FIG. 14G) over this time. This stability study was repeated using lyophilised CNs prepared in the same buffer before storing at 25° C. while protecting from light. The residual water content calculated at 120° C. was 1.085±0.050%, indicating lyophilized formulations were properly prepared. Formulations were resuspended prior to recording dispersion properties (FIG. 14E-G) which were found to remain constant and similar to those reported for liquid formulations (Table 7). EE % was found to decline by an average of 20% versus baseline at each time point assessed, suggesting this may be a result of the lyophilization or rehydration process.

Several groups have previously attempted to prepare curcumin loaded nanoparticle formulations, including; PLGA-nanocarriers, solid lipid nanocarriers, liposomes and exosomes. Existing nanoparticulate formulations of curcumin possess limited stability (not assessed beyond 72 h in any study cited), only moderate curcumin loading has been achieved (<0.77 mg/mL) and most protocols would be difficult to translate to the clinic owing to complex, multi-step manufacture protocols requiring organic solvents. The TPGS/Pluronic F127 curcumin formulation described here compares favourably with those in the existing literature.

TABLE 7
Characteristics of curcumin loaded nanocarriers and stability over time (n = 3).

| | Mean (SD) | | | | | | | |
|---|---|---|---|---|---|---|---|---|
| | BASELINE | | THREE WEEKS | | SIX WEEKS | | NINE WEEKS | |
| Storage | 25° C. | Lyophilized | 25° C. | Lyophilized | 25° C. | Lyophilized | 25° C. | Lyophilized |
| EE (%) | 94.2 (4.1) | 101.6 (6.7) | 92.6 (1.7) | 81.2 (9.7) | 97.1 (1.1) | 87.8 (1.6) | 91.0 (2.2) | 78.2 (7.4) |
| Z-Diameter | 17.9 (0.4) | 18.7 (0.5) | 17.8 (0.5) | 19.3 (2.1) | 17.7 (1.0) | 17.9 (1.6) | 17.3 (0.1) | 18.5 (0.5) |
| (nm) | | | | | | | | |
| PDI | 0.002 (0.004) | 0.128 (0.029) | 0.146 (0.027) | 0.183 (0.060) | 0.168 (0.046) | 0.218 (0.038) | 0.188 (0.001) | 0.177 (0.0011) |

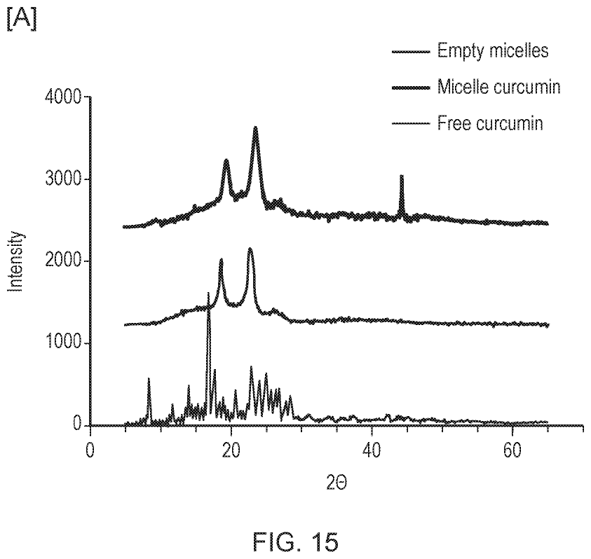

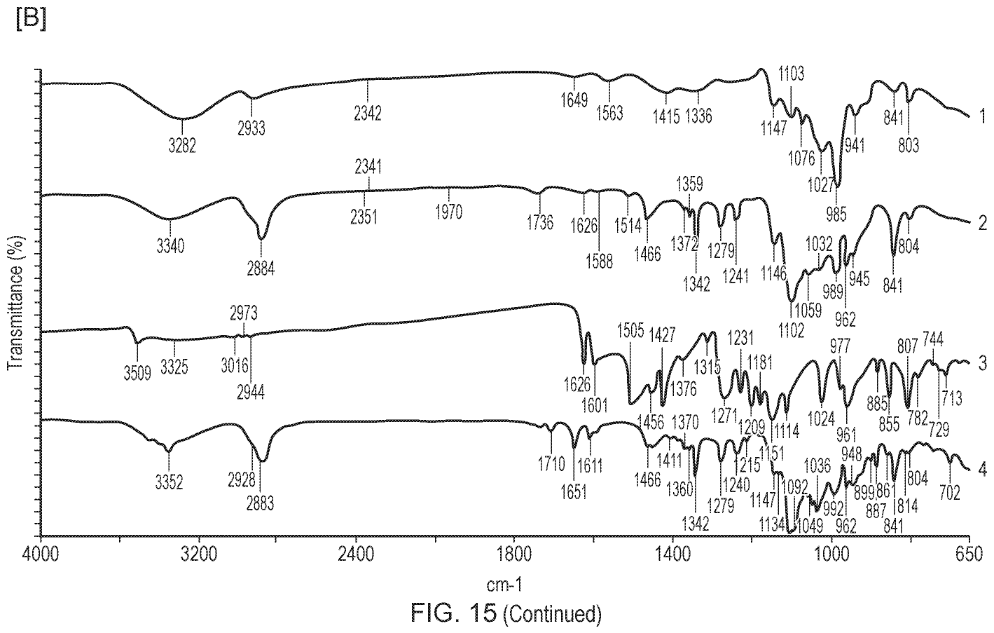

XRD and FT-IR spectra were acquired to ascertain the nature of curcumin once incorporated into nanocarriers (FIG. 15). The X-ray diffraction patterns of free curcumin exhibited characteristic peaks between 5° and 30°, indicative of a highly crystalline structure. This character was lost on inclusion of curcumin in a nanocarrier formulation, indicating that curcumin has successfully been incorporated into the amorphous nanocarrier structure and is not associated with the particle surface. FT-IR spectra reveal characteristic peaks of free curcumin at 3509 cm⁻¹, 1626 cm⁻¹, 1601 cm⁻¹, 1505 cm⁻¹, 1271 cm⁻¹, 1024 cm⁻¹, 948 cm⁻¹ and 713 cm⁻¹ which closely match previously reported values. On incorporation into nanocarriers, the characteristic curcumin peak at 3509 cm⁻¹ (indicative of the free hydroxyl group) merged with the broad OH peak of the TPGS/Pluronic F127 carrier at 3352 cm⁻¹, which may suggest complex formation. Furthermore, characteristics shifts in the aromatic C═C peak (1601 cm⁻¹ to 1588 cm⁻¹) and the C=0 stretching, δ(CCC) and δ(CCO) in plane bending from 1505 cm⁻¹ to 1514 cm⁻¹ have previously been interpreted as evidence for the successful incorporation of curcumin into a complex.

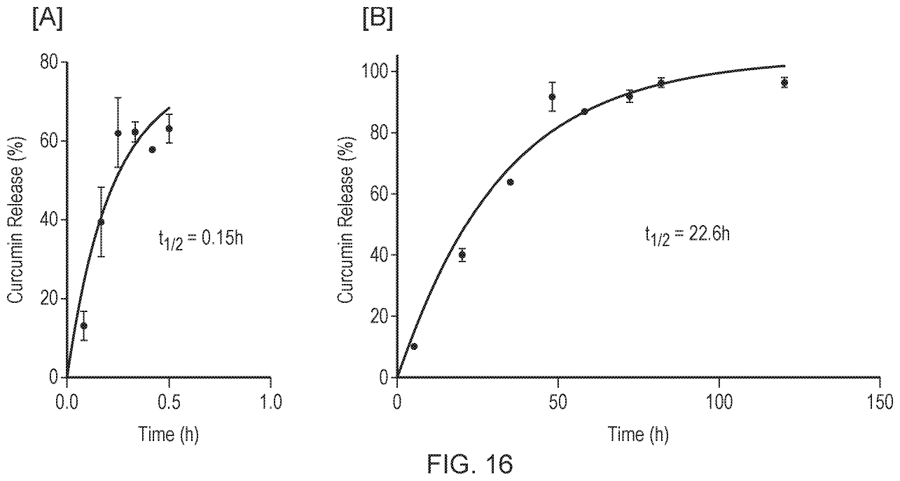

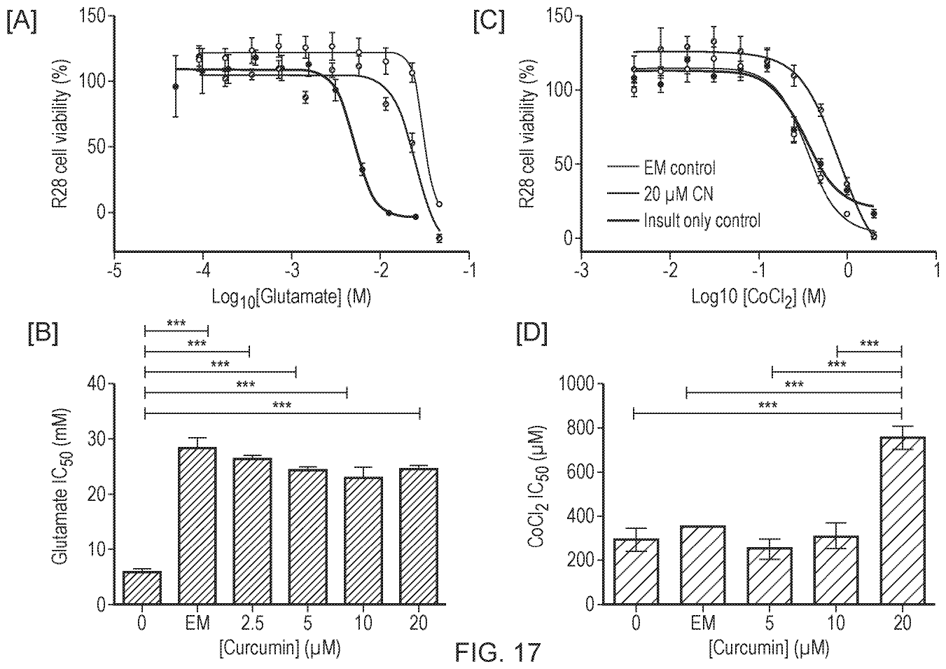

Formulation of curcumin into nanocarriers substantially reduced the rate of drug release compared to free drug (t_{1/2}=22.6 h versus 0.15 h respectively, FIG. 16) at 37° C., attributed to the slow rate of release of curcumin from nanocarriers. Less than 10% of the drug was liberated after 5 h of incubation, suggesting that there was little burst release from the CN formulation. This observation supports FT-IR and XRD observations that curcumin is not merely associated with the nanocarrier surface but is localised within the hydrophobic interior in an amorphous or disordered crystalline phase, in agreement with previous work. Together, these results suggest that the curcumin-loaded nanocarrier formulation described in this study have sustained release capability.

### Curcumin-Loaded Nanocarriers Protect a Retinal Cell Line Against Glutamate and Hypoxia-Induced Injury

Glutamate excitotoxicity represents a potential mechanism leading to RGC loss in glaucoma. Using an AlamarBlue cell viability assay, co-incubation of immortalised R28 cells with both CNs and empty nanoparticles was found to be significantly protective (one-way ANOVA with Tukey post-test, p<0.001) against glutamate induced toxicity (FIGS. 17A & B, IC₅₀ 28.3±3.4 mM versus 5.9±1.2 mM for EM and insult only treated groups respectively, on-way ANOVA with Tukey post-test p<0.001) with no additive effect observed on addition of curcumin to the nanoparticles (24.5±1.2 mM, CN containing 20 μM curcumin). This observation is in agreement with previous studies that suggest α-tocopherol (here present in the form of TPGS) is protective against glutamate induced toxicity and this has been suggested to be a result of the anti-oxidant function of this molecule. As TPGS was not also protective against cobalt chloride induced insult, this suggests curcumin and TPGS may have additive therapeutic effects.

Upregulation of hypoxia-related factors such as Hypoxia Inducible Factor 1α (HIF-1α) has been suggested to implicate hypoxia in glaucoma pathology. Cobalt chloride (CoCl₂) is a hypoxia mimetic and inducer of HIF-1α 75 used as an in vitro glaucoma model. The IC₅₀ of R28 cells exposed to CoCl₂ for 24 h (FIGS. 17C & D) was found to be significantly increased on concurrent incubation with 20 μM curcumin in the form of CN (296±53 μM vs 757±51 μM respectively, one-way ANOVA with Tukey post-test, p<0.001). Treatment with an equivalent concentration of the nanoparticle in the absence of curcumin had no significant effect (296±53 μM versus 354±8 μM, one-way ANOVA with Tukey post-test, p>0.05) suggesting that the protective effects observed were as a result of curcumin. Concentrations of curcumin <20 μM were not found to be neuroprotective in this model. Curcumin has previously been reported to inhibit HIF-1α in hepatocellular carcinoma cells and was more recently reported to supresses HIF-1α synthesis in pituitary adenomas. HIF-1α inhibitors have previously been proposed as potential glaucoma treatment worthy of further investigation.

Topically administered curcumin nanocarrier therapy protects RGCs in rodent models of ocular hypertension and optic nerve injury Having established the neuroprotective activity of CNs in vitro in relation to vehicle only treatments, we next assessed the neuroprotective effects of this formulation on RGC health using an established in vivo rodent model of RGC loss. We anticipate that topically applied curcumin loaded nanoparticles will reach the retina via a combination of topical and systemic absorption routes. In support of this hypothesis, Sigurdsson et al reported that their formulation of dexamethasone, which is a similar molecular weight to curcumin (392 versus 368 Da respectively), entered the retina 60% via topical penetration and 40% by systemic absorption route. We anticipate that the well-documented P-gp inhibition activity of tocopherols and curcumin, in conjunction with enhanced corneal penetration activity previously reported for PEGylated-micelle formulations will enhance curcumin delivery to the retina by the topical absorption route.

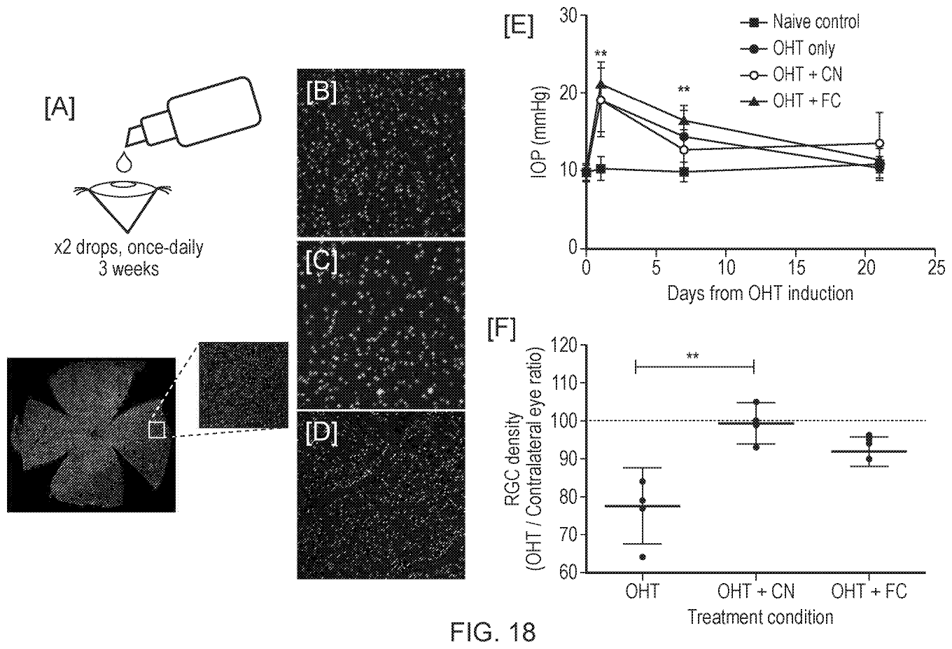

Optimum time points post model induction (maximal RGC loss in shortest time after induction) were chosen based on our previous work characterising the natural history of the OHT and pONT models where multiple time points were assessed after model induction. We recently reported that administration of TPGS containing micelles did not themselves have a neuroprotective effect in vivo, which in conjunction with our in vitro observations, suggest that any neuroprotective efficacy observed was a result of curcumin treatment. Rats received topical CNs according to the dosing regimen illustrated in FIG. 18A. Briefly, two days prior to OHT model induction, rodents began receiving two drops (35 μL each) of CNs dosed five minutes apart per day for three weeks from the date of model induction. Topical administration of CNs was found to be well-tolerated by rats with no signs of ocular irritation or inflammation reported in naive eyes monitored by a qualified ophthalmologist. The IOP profile of rodent's after IOP elevation by injection of hypertonic saline into two episcleral veins (FIG. 18B) indicates that IOP remained significantly elevated for at least 7 days after model induction versus naive eyes. No significant difference in IOP profile between CN and OHT only groups was observed, suggesting that any neuroprotective effect of curcumin was due to IOP independent processes. RGC health was assessed histologically from whole-retinal mounts using brn3a assessment (FIG. 18C). This approach was chosen as Brn3a is a nuclear restricted and RGC specific transcription factor that exclusively label 97% of the RGC population (excluding photosensitive RGCs). We have also recently developed an algorithm to accurately and automatically quantify whole RGC populations in rodent models of retinopathy enabling the reliable assessment of RGC health. Using this approach, OHT induction was found to result in a significant reduction in global RGC density of ˜23% compared to contralateral eyes, which is comparable to previous studies using this model. CN administration significantly improved the RGC density ratio between OHT eye vs contralateral untreated eyes (Kruskal-Wallis test with Dunns post test, **p<0.01), whereas administration of un-encapsulated curcumin (FC—free curcumin) solubilised in PBS did not have this effect (FIG. 18D-F).

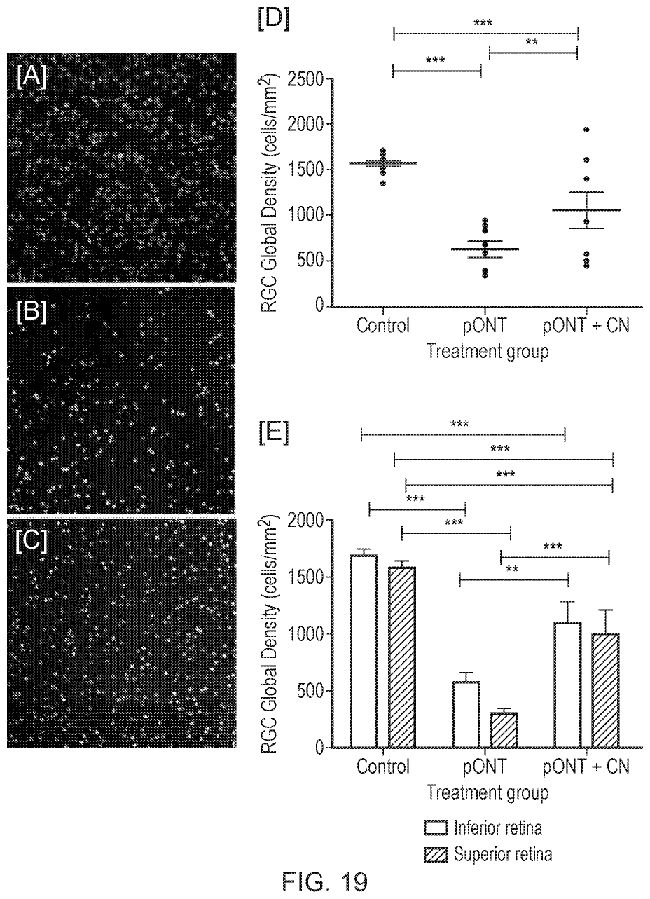

To further investigate the neuroprotective potential of topically applied CNs, whole-retinal brn3a labelled RGC population assessments were made in the pONT model (FIG. 19A). In this model, twice-daily topical administration of CNs was found be significantly protect RGCs (one-way ANOVA, ***p<0.001). On subdivision of whole retinal mounts into superior and inferior quadrants (FIG. 19B), treatment with CNs was observed to result in preservation of RGC populations in both the superior and inferior quadrants, but this effect was more pronounced in the superior retina (two-way ANOVA, ***p<0.001), which may imply the protective effects of curcumin therapy exert through anti-apoptotic as well as anti-oxidant mechanisms. Representative regions from the superior quadrant of Brn3a labelled retinal whole-mounts (FIG. 19C-E) illustrate RGC populations were diminished in retina subject pONT (FIG. 19D) versus naive controls (FIG. 19C). Treatment with CNs for three weeks was found to protect RGC soma from pONT induced injury (FIG. 19E). As preservation of RGC soma was observed in both the superior and inferior retinal quadrants, this suggests that curcumin may elicit neuroprotective activity via multiple pathways involving both primary and secondary neurodegeneration processes.

The possibility of TPGS mediated neuroprotection via inhibition of glutamate excitotoxicity is intriguing and may contribute to the neuroprotective effect of our formulation in vivo. In support of this hypothesis and our present in vitro findings, Nucci et al previously reported that intraocular administration of a total of 10 μL of 0.5% (w/v) TPGS (equivalent to a total dose of 0.5 mg TPGS) was neuroprotective against ischemia/reperfusion injury in the rat. Previously, we reported that topical administration of TPGS at the same concentration did not have a neuroprotective effect in vivo. This discrepancy is likely to the lower concentration reaching the retina compared to invasive application, typically estimated to be ˜3% of the topically applied dose. Although our previous work with this model suggests that administration of TPGS only did not appear to have a neuroprotective effect in its own right, a synergism between curcumin and TPGS is extremely likely, if not via the neuroprotective effects of TPGS alone, then perhaps via TPGS mediated modulation of P-gp activity, enhancing curcumin transport across ocular barriers.

The neuroprotective effect of curcumin loaded nanocarriers observed in this study may be a result of treatment commencing two days before model induction, suggesting this therapy may be most effective for patients at risk of IOP spikes such as following phacoemulsification surgery or as a prophylactic to patients identified at high risk of developing glaucoma such as those with ocular hypertension or other glaucoma risk factors. Furthermore, with the development of new techniques such as DARC (detection of apoptotic retinal cells) with the potential to diagnose glaucoma earlier in the disease process (Cordeiro et al. 2017), therapies to slow or prevent RGC loss at earlier stages of disease progression will play a greater role in glaucoma management.

In conclusion, this study describes a novel nanocarrier formulation of curcumin in TPGS/Pluronic F127 that increases the solubility of this poorly soluble drug by a factor of almost 400,000. This formulation incorporates 4.3 mg/mL of curcumin with an encapsulation efficiency consistently >90% and excellent stability in liquid or lyophilized forms for at least two months when stored at room temperature, as determined by HPLC and spectroscopic techniques. This formulation was found to be neuroprotective against glutamate and cobalt chloride induced injury in retinal cultures in vitro and significantly preserved RGC density in two well-established rodent models of ocular injury. In conclusion, we demonstrate that curcumin loaded nanoparticles have exciting potential for overcoming ocular barriers and may facilitate the translation of curcumin based therapies to the clinic for the treatment of ocular conditions such as glaucoma.

### Example 3—Acylhomoserine Lactones (AHSLs) are Enhancers of Drug Delivery

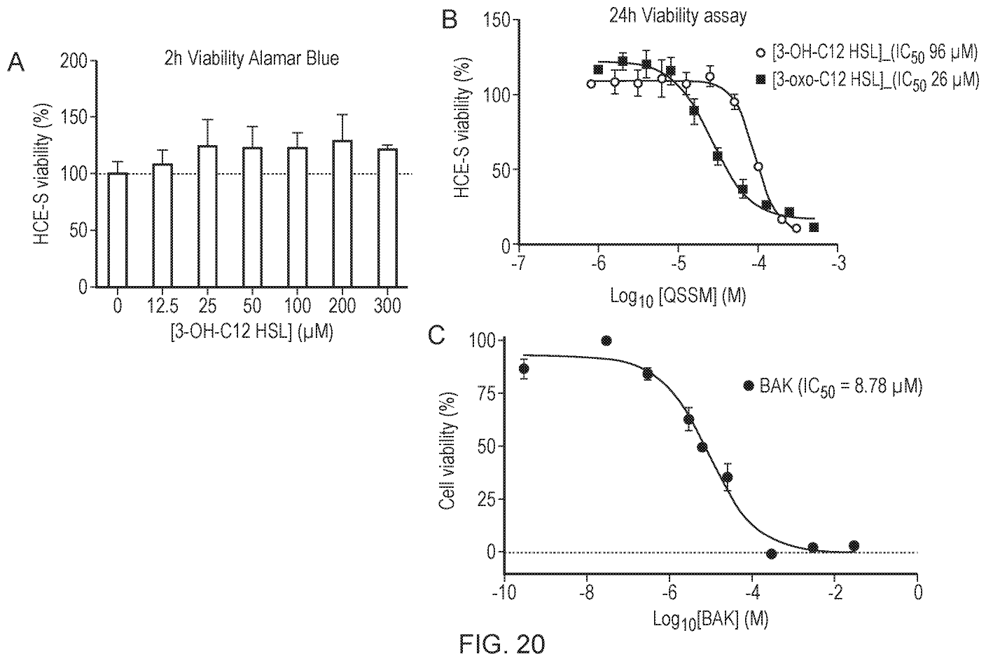

Turning to FIGS. 20 to 23, these results demonstrate the safety and efficacy of AHSLs as drug delivery enhancers in a composition according to the additional aspect of the invention defined above. The results demonstrate enhanced transepithelial delivery of two structurally unrelated model agents, FITC-dextran and FITC-Annexin V, and an antibody, bevacizumab, in AHSL-containing micelles. The micelles (or other ternary formulation systems) can be prepared by a variety of methods, as would be appreciated by the skilled person. However, thin film hydration can be mentioned, as can the simple mixing of the formulation components in an aqueous medium, with optional warming.

### Preparation of Exemplary Compositions

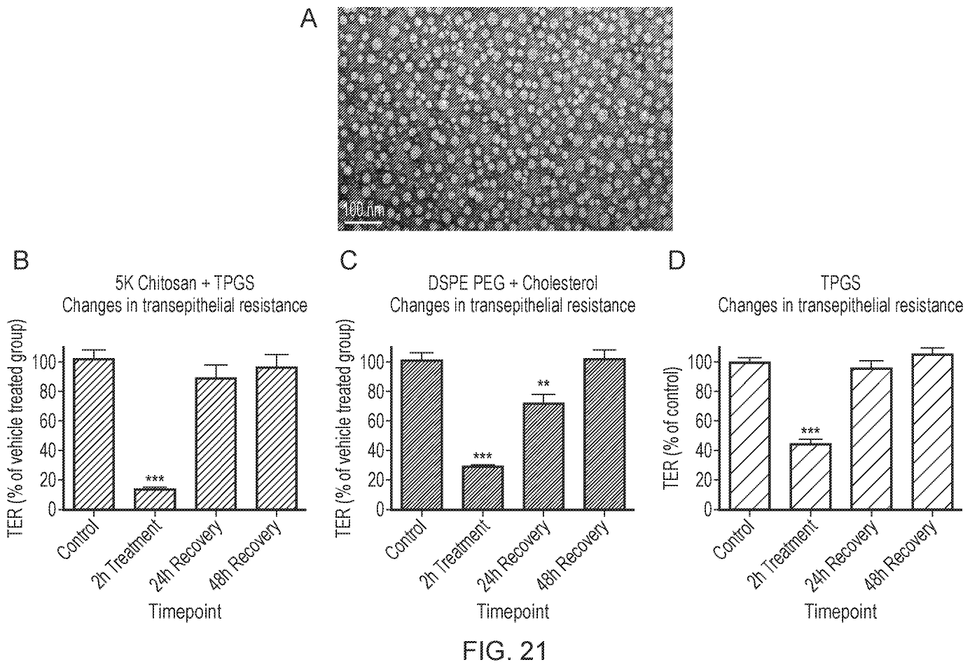

In further detail, and by way of example only, the compositions can be prepared as follows (note that Formulations 1 to 3 are reported in FIG. 21):

Formulation 1. 3-OH—C12 HSL with TPGS or DSPE-PEG and Cholesterol Micelles
a. 100 mg of TPGS (or DSPE-PEG and cholesterol) and 5 mg·mL⁻¹ of 3-OH—C12 HSL was solubilised in 5:1 chloroform:methanol
b. The desired quantity of TPSG (or DSPE-PEG and cholesterol) and 3-OH—C12 HSL (typically 0.5% and 0.1% w/v respectively) were aliquoted and solvent evaporated by rotary evaporation (65° C., 50 mBar for 1 h)
c. Cake was resuspended in PBS buffer at 65° C. for 30 minutes before filtration (0.22 μm) to remove unencapsulated material.
   Formulation 2. 3-OH—C12 HSL with TPGS Micelles
a. TPGS powder was solubilised directly into PBS buffer by heating to 65° C. for 2 h to a final concentration of 0.5% (v/v) to produce TPGS micelles
b. Alternatively to a, TPGS micelles were prepared by sonication of the solution in A
c. 3-OH—C12 HSL was solubilised in DMSO to a concentration of 10 mg/mL before 0.1% (v/v) was mixed with the TPGS micelle solution for 1 h
d. Unencapsulated material was removed by 0.22 μm filtration.
   Formulation 3. 3-OH—C12 HSL with TPGS/5 k Chitosan Micelles
a. 55 mg/mL 5K Chitosan was prepared by dissolving 110 mg of 5K Chitosan in 2 mL of buffer (10 mM HEPES, 140 mM sodium chloride, pH 7.4) on heating to 37° C. for 1 h.
b. A TPGS solution was prepared as described above (F1 or 2)
c. 25 mg of 5K chitosan was combine with 2.75 mg of TPGS from above solutions and added to 0.1 mg of 3-OH—C12 HSL and incubated at 37° C. for 1 h with brief sonication to fully dissolve
d. Remove unencapsulated material by filtration (0.22 μm pore)

### Formulation 4. 3-OH—C12 HSL Loaded Liposomes

1. Prepare the following formulation of PC_{50%} PS_{15%} Cholestero_{130%}QSSM_{5%}PLVs (65 mM) with 3.25 mM QSSM by dissolving in 5:1 Chloroform:Methanol.
   PC_{70%} (25 mg/ml): 1001 μl
   PS_{15%} (25 mg/ml): 322 μl
   Cholesterol (50 mg/ml): 151 μl
   3-OH—C₁₂ HSL (5 mg/ml): 972 μl
2. Evaporate under argon and then under vacuum for 1 h to produce a dried lipid cake
3. Meanwhile dialyse 1 ml of AnxV-776 (13-03, 0.475 mg/ml) against 10 mM HEPES, 140 mM NaCl pH 6.5 for 3 h
4. Rehydrate the lipid cake in 1 ml AnxV-776 solution for 1 h incubating at 37° C. with gentle mixing (150 rpm)
5. Freeze-thaw mixture 5 times in liquid nitrogen
6. Extrude sequentially using 400 nm, 200 nm and finally 100 nm pore filters

Note that in these exemplary compositions, 3-OH—C12 HSL can be substituted with any AHSL.

### Transwell Assay Protocol (Based in Part on Davis et al. (2014))

HCE-S cells (Immortalized Human Corneal Epithelial Cells) were cultured as a monolayer in 90% DMEM supplemented with 10% heat inactivated FBS and penicillin-streptomycin (100 U/mL). Cells were cultured at 37° C. in a humidified incubator with 5% CO₂ and growth medium was replaced every three days. HCE-S populations were grown to 80% confluence before passage to 20% using 0.25% Trypsin-EDTA. Prior to each experiment cell populations were estimated using a haemocytometer and trypan blue exclusion assay.

HCE-S barrier models for transcytosis assays were prepared using an adaptation of a previously described method (Reichl (2008)). HCE-S cells (5×10⁴ cells/well) were seeded on transwell inserts (polycarbonate, 3 μm pore size, 1.13 cm²) and maintained for 7 days refreshing the medium in the apical and basal chambers every second day. After this time cells were cultured for a further 10 days at an apical air interface to encourage development of a multilayer barrier. Prior to each experiment culture medium was replaced with phenol red free DMEM and barrier integrity measured via transepithelial resistance (TER). TER values were obtained in the range 300-600 Ωcm² and only epithelial barriers with TERs >300 Ω·cm², the accepted threshold for tight epithelial barriers, were used for transcytosis experiments (Becker et al.).

FIGS. 21 & 22. Baseline TER was recorded before addition of 300 μM 300 3-OH—C12 HSL in micelles (assuming 100% drug loading) or equivalent concentrations of micelles only (vehicle control) dissolved in phenol-red free culture medium for 2 h. After this time, TER was recorded before culture medium was replaced with fresh. To the apical chamber, a model drug molecule (FITC-dextran or Avastin) was added and the concentration of this agent in the basolateral chamber was measured either by monitoring FITC fluorescence (FITC-dextran) versus a standard curve of known concentrations or using a commercially available Avastin sandwich ELISA kit as previously described (Davis et al. (2014)). TER measurements at each timepoint were normalised to vehicle treated controls and clearly demonstrate the ability of AHSLs to reversibly disrupt biological barriers and importantly, the exploitation of this concept for drug delivery applications.

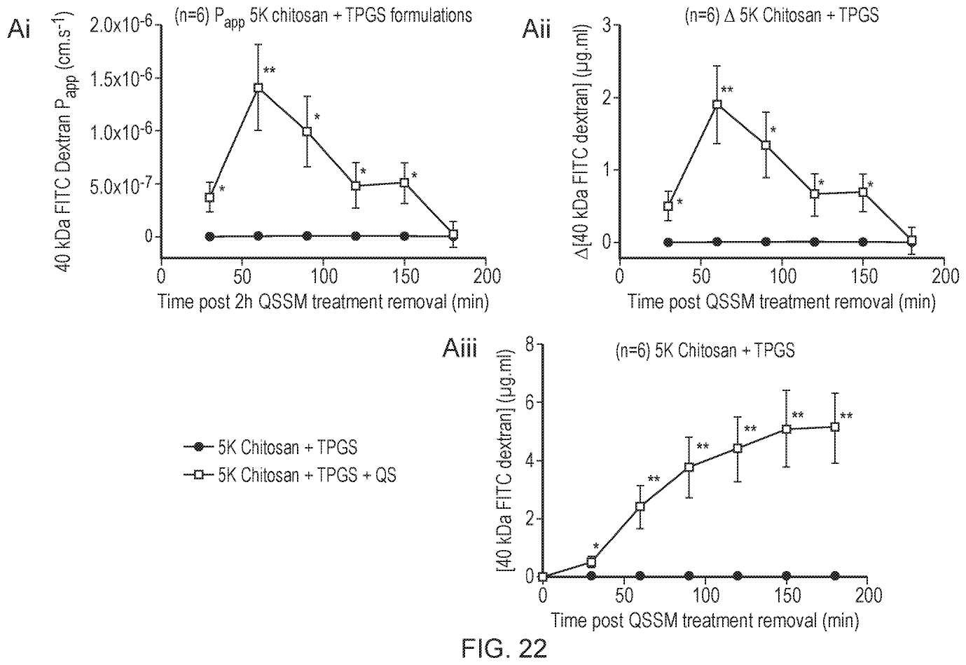

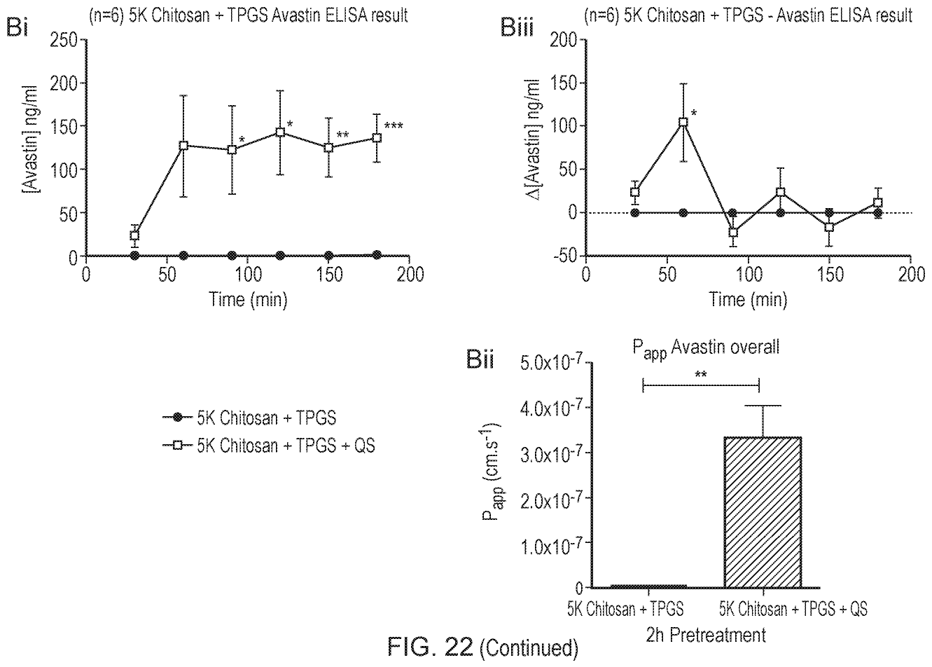

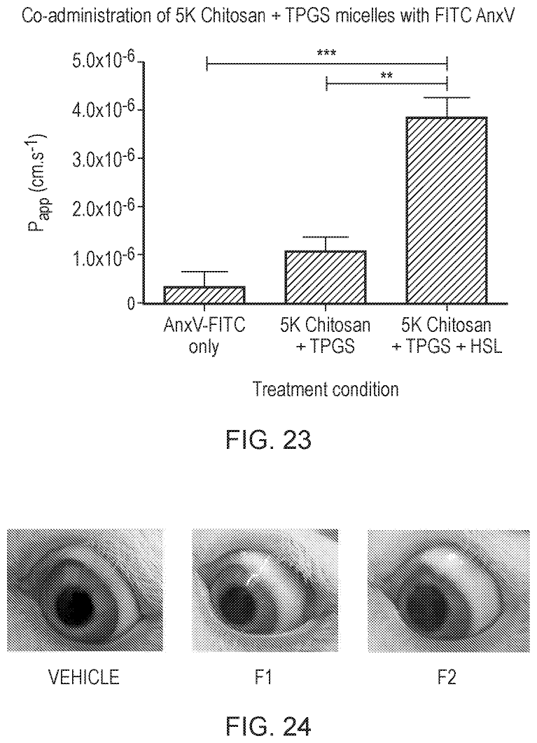

FIG. 23. FITC ANXV (a model protein drug in this instance) was co-administered with AHSL loaded micelles for 3 h to demonstrate that co-administration of AHSL containing formulations as well as pre-treatments could be utilised to enhance delivery of agents with therapeutic potential across biological barriers. Protocol as described in connection with FIGS. 21 and 22.

Although the HCE-S transwell model used here is a model of the corneal epithelium, the method of acylhomoserine lactone-mediated reversible barrier disruption is via interaction with IQGAP1 [Karlsson et al.]. A similar effect will therefore be observed in other biological barriers (i.e. blood-brain, blood retina, intestinal and dermal barriers).

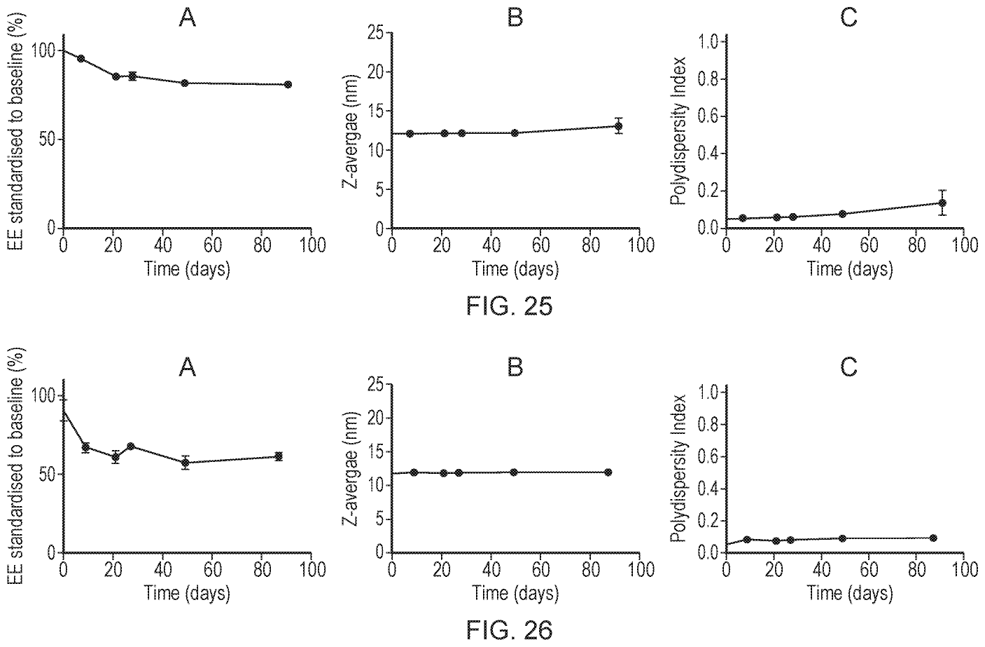

To date the ability of acylhomoserine lactones to disrupt biological barriers has focused solely on the ability of these agents (which are produced by bacteria, and 3-OH—C12 HSL is a synthetic analogue of 3-oxo-C12 HSL) to permit bacteria to invade host tissues. This work presents the first demonstration of the use of these agents to facilitate the delivery of therapeutics (including macromolecules such as polysaccharides and large proteins (FIGS. 25 and 26), but the skilled person would appreciate that the concept would also work for small molecule APIs) across biological barriers.

### Example 4—HET-CAM Test

### Materials and Methods

To study the ocular tolerance in vitro the HETCAM® test was developed as described in the INVITTOX no15 protocol (Warren et al. 1990).

This test is based on the observation of the irritant effects (bleeding, vasoconstriction and coagulation) in the chorioallantoic membrane (CAM) of a 10 days embryonated egg induced by application of 0.3 ml of each of the studied formulations for the first 5 min of its application.

These eggs (from the farm G.A.L.L.S.A, Tarragona, Spain) were kept at a temperature of 12±1.0 for at least 24 h before placing them in the incubator with controlled temperature (37.8° C.) and humidity (50-60%) during the incubation days.

A series of controls were performed: SDS 1% (positive control for slow irritation), 0.1 N NaOH (positive control for fast irritation), NaCl 0.9% (negative control).

Data were analysed as the media±SD of the time at which the injury occurred (n=3/group). Scores of irritation potential can be grouped into four categories (Table 8).

TABLE 8
HET-CAM calculation and classification

| Calculation of OII (HET-CAM) | OII | Classification |
|---|---|---|
| OII = (301 − h) · 5/300 + | 0-0.9 | Non-irritant |
| (301 − v) · 7/300 + | 1-4.9 | Weakly irritant |
| (301 − c)*9/300 | 5-8.9 | Moderately irritant |
| | 9-21 | Irritant |

H: haemorrhage,
v: vasoconstriction,
C: coagulation

### Results

The formulations analysed showed to be non-irritant in vitro (Table 9). Interestingly, in all the cases the only phenomena that appeared was a slight vasoconstriction process (FIG. 24). In none of the formulations hemorrhage or coagulation were present.

TABLE 9
Results of the formulations assessments

| | H | V | C | OII | OII mean |
|---|---|---|---|---|---|
| Vehicle | — | — | — | 0 | 0.66 |
| Vehicle | — | 267 | — | 0.79 | |
| Vehicle | — | 248 | — | 1.2 | |
| F1 | — | 180 | — | 2.82 | 0.94 |
| F1 | — | — | — | 0 | |
| F1 | — | — | — | 0 | |
| F2 | — | 199 | — | 2.38 | 0.79 |
| F2 | — | — | — | 0 | |
| F2 | — | — | — | 0 | |

### In Vivo

### Materials and Methods

In vivo ocular tolerance was assessed using the Draize irritation test. It was performed using New Zealand albino male rabbits of 2.5 kg middle weight from San Bernardo farm (Navarra). This test was performed according to the Ethical Committee for Animal Experimentation of the UB and current legislation (Decret214/97, Gencat).

The sample was placed in the conjunctival sac of the left eye and a gentle massage was applied to assure the proper circulation (Nobrga et al. 2012). The appearance of irritation was observed both at the time of administration and after 1 h, using the right eye as a negative control (n=3/group).

The evaluation was performed by direct observation of the anterior segment of the eye, noting the possible injury of the conjunctiva (inflammation, chemosis, redness or oozing), iris and cornea (opacity and affected surface). Ocular irritation index (OII) was evaluated according to the observed injuries (Tables 10 and 11).

TABLE 10
Draize test calculation and classification

| Calculation of OII (Draize test) | OII | Classification |
|---|---|---|
| OII = Corneal (A · B · 5) + | 0-0.9 | Non-irritant |
| Iris (A · 5) + Conjunctiva (A + | 1-4.9 | Weakly irritant |
| B + C)*2 | 5-8.9 | Moderately irritant |
| | 9-21 | Irritant |

TABLE 11
Draize test evaluation score.

| Structure | Injury | Evaluation | Score |
|---|---|---|---|
| Cornea | A) Degree of cloudiness | | Corneal |
| | or opacity | | score: |
| | Absence of ulceration | 0 | A · B · 5 |
| | Diffuse areas | 1 | |
| | Translucent areas | 2 | |
| | Opalescent areas | 3 | |
| | Full opacity | 4 | |
| | B) Affected area | | Maximum |
| | None | 0 | score: |
| | A quarter or less | 1 | 80 |
| | More than a quarter but | 2 | |
| | without means | | |
| | More than half but less | 3 | |
| | than three quarters | | |
| | More than three quarters | 4 | |
| | up a whole plane | | |
| Iris | A) Iris injury score | | Radial score: |
| | Normal | 0 | A · 5 |
| | Deep folds, congestion, | 1 | Maximum |
| | swelling, moderate | | score: 10 |
| | circumcorneal injection. | | |
| | No reaction to light, | 2 | |
| | hemorrhage, great destruction | | |
| Conjunctiva | A) Redness | | Conjunctival |
| | Normal glasses | 0 | score: |
| | Some clearly injected vessels | 1 | (A + B + |
| | Diffuse redness | 2 | C) · 2 |
| | Big diffuse redness | 3 | |
| | B) Chemosis or Inflammation | | Maximum |
| | None | 0 | score: 20 |
| | Some | 1 | |
| | Marked with partial disorder | 2 | |
| | of the eyelids | | |
| | Eyelid more or less closed | 3 | |
| | Semi eyelids | 4 | |
| | C) Sweat | | |
| | None | 0 | |
| | Any amount anomalous | 1 | |
| | Wetting and eyelid hairs | 2 | |
| | Periocular wetting | 3 | |

### Results

None of the formulations was irritant (011=0). The animals did not show any sign of irritation in vivo at the time of the application or after one hour (FIG. 24). This correlates with the results of the in vitro assessment where the products show to be non-irritant.

### Example 5

A formulation of 4.5 mg/ml curcumin encapsulated in micelles formed from 25 mg/ml TPGS and 150 mg/ml solutol was prepared using the previously described thin-film rehydration technique.

The formulation demonstrated high encapsulation efficiency and stability over 90 days (FIG. 25 A-C). When freeze-dried and subsequently rehydrated, encapsulation efficiency remained high (65%), while particle size and polydispersity also demonstrated stability over 90 days (FIG. 26 A-C).

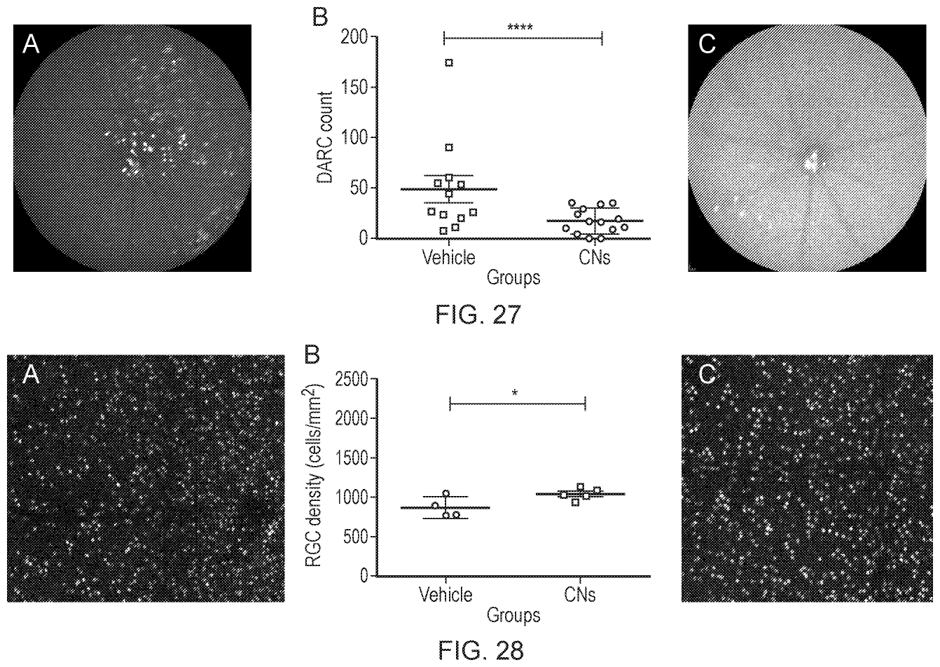

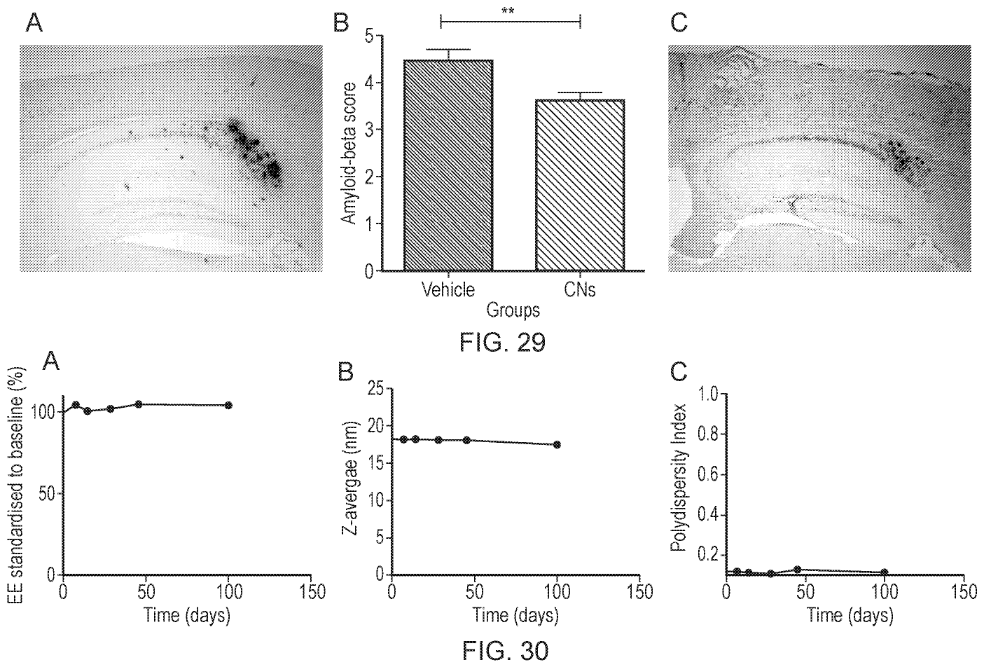

The formulation was then tested in the 3xTg-AD mouse model of Alzheimer's disease and was administered intranasally, 5 days per week for 3 months. The curcumin nanoparticles decreased the DARC count (see e.g. WO 2011/055121 for further details of DARC count) in the retina when compared to vehicle alone, indicating that cell death was reduced (FIG. 27 A-C). The curcumin nanoparticles also protected retinal ganglion cells (RGCs) from loss (FIGS. 28 A-C) and reduced amyloidβ deposition in the hippocampus (FIGS. 29 A-C).

### Example 6

A formulation of 15 mg/ml resveratrol encapsulated in micelles formed from 25 mg/ml TPGS and 150 mg/ml solutol was prepared using the previously described thin-film rehydration technique.

The formulation demonstrated high encapsulation efficiency (>70%) and stability over 90 days (FIGS. 27 A-C). When freeze-dried and subsequently rehydrated, encapsulation efficiency remained high, while particle size and polydispersity also demonstrated stability over 90 days (FIGS. 28 A-C).

R28 cells were cultured as described above before being treated with resveratrol (20 μm) containing micelles or an equivalent concentration of TPGS/solutol only (i.e., empty) micelles, in conjunction with varying concentrations of cobalt chloride or glutamate insults.

The resveratrol containing micelles were observed to be neuroprotective against glutamate excitotoxicity (FIG. 29 A-D) but were not neuroprotective against cobalt chloride induced hypoxia (FIG. 30 A-D).

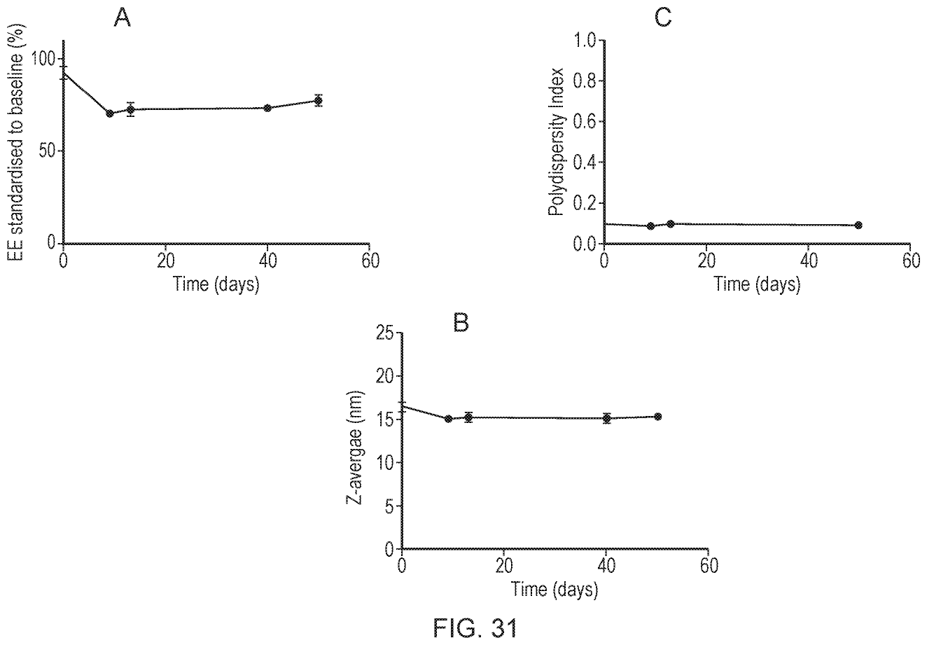

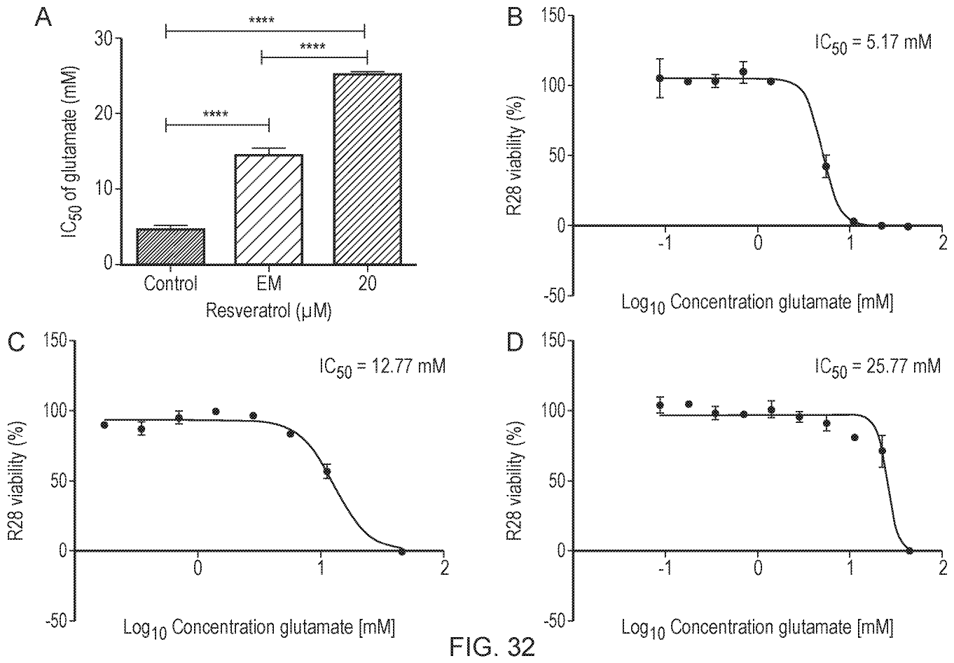

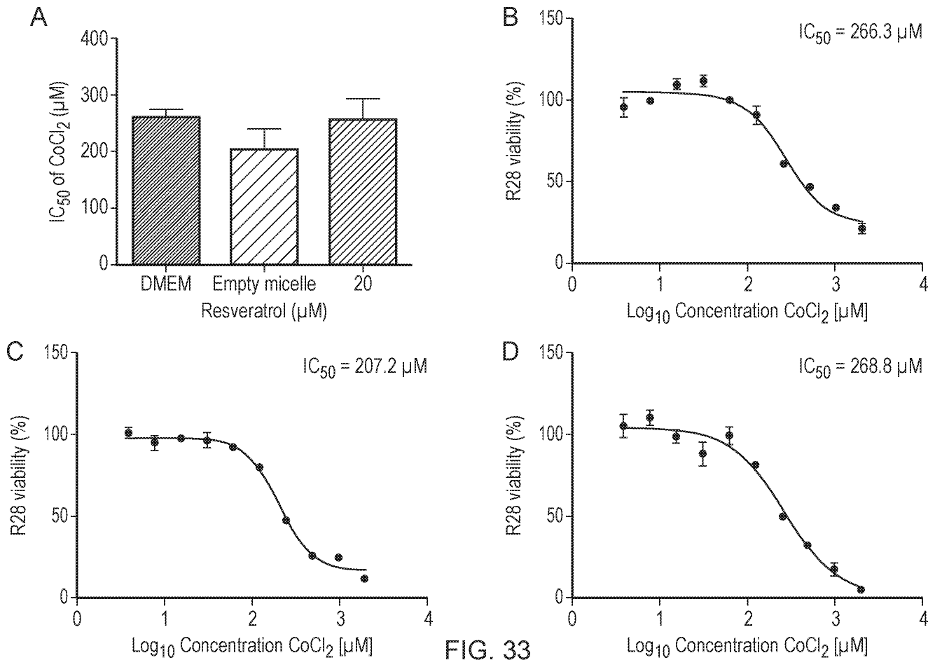

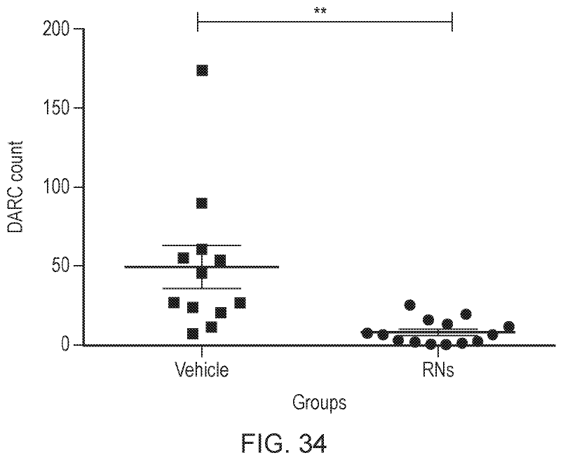

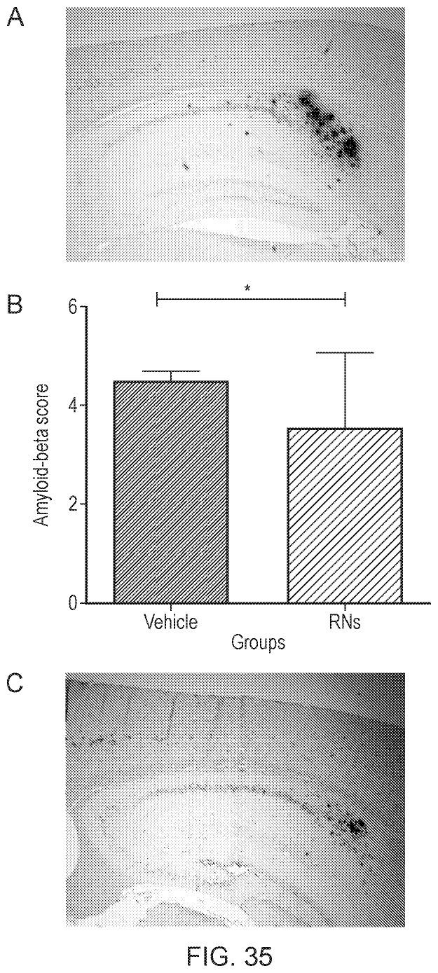

The formulation was then tested in the 3xTg-AD mouse model of Alzheimer's disease and was administered intranasally, 5 days per week for 3 months. The resveratrol nanoparticles decreased the DARC count in the retina when compared to vehicle alone, indicating that cell death was reduced (FIG. 31) and reduced amyloidβ deposition in the hippocampus (FIG. 32 A-C).

### REFERENCES

Becker U, Ehrhardt C, Daum N, Baldes C, Schaefer U F, Ruprecht K W, Kim K J, Lehr C M (2007) Expression of ABC-transporters in human corneal tissue and the transformed cell line, HCE-T. J Ocul Pharmacol Ther. 23(2):172-81
Biehlmaier O, Alam M, Schmidt W J (2007) A rat model of Parkinsonism shows depletion of dopamine in the retina. Neurochemistry international 50: 189-195 Doi 10.1016/j.neuint.2006.08.001
Bizrah M, Dakin S C, Guo L, Rahman F, Parnell M, Normando E, Nizari S, Davis B, Younis A, Cordeiro M F (2014) A semi-automated technique for labeling and counting of apoptosing retinal cells. BMC bioinformatics 15:169 Doi 10.1186/1471-2105-15-169
Cooley M A, Whittall C, Rolph M S (2010) Pseudomonas signal molecule 3-oxo-C12-homoserine lactone interferes with binding of rosigliatzone to human PPARγ. Microbes and Infection 12 231-237
Cordeiro M F, Guo L, Luong V, Harding G, Wang W, Jones H E, Moss S E, Sillito A M, Fitzke F W (2004) Real-time imaging of single nerve cell apoptosis in retinal neurodegeneration. Proc Natl Acad Sci USA 101: 13352-13356 Doi 10.1073/pnas.0405479101
Cordeiro, M. F. et al. Real-time imaging of single neuronal cell apoptosis in patients with glaucoma. Brain 274, 61-5 (2017).
Davis, B. M., Richens, J. L. & O'Shea, P. Label-free critical micelle concentration determination of bacterial quorum sensing molecules. Biophys. J. 101, 245-54 (2011).
Davis B M, Normando E M, Guo L, Turner L A, Nizari S, O'Shea P, Moss S E, Somavarapu S, Cordeiro M F (2014) Topical delivery of Avastin to the posterior segment of the eye in vivo using annexin A5-associated liposomes. Small 10(8):1575-84.
Davis B M, Guo L, Brenton J, Langley L, Normando E M, Cordeiro M F (2016) Automatic quantitative analysis of experimental primary and secondary retinal neurodegeneration: implications for optic neuropathies. Cell Death Discovery 2, Article number 16031 (2016) doi:10.1038/cddiscovery.2016.31
Galvao J, Davis B, Tilley M, Normando E, Duchen M R, Cordeiro M F (2014) Unexpected low-dose toxicity of the universal solvent DMSO. FASEB Journal: official publication of the Federation of American Societies for Experimental Biology 28: 1317-1330 Doi 10.1096/fj.13-235440
Guddadarangavvanahally K. Jayaprakasha, Lingamullu Jagan Mohan Rao, and & Sakariah*, K. K. Improved HPLC Method for the Determination of Curcumin, Demethoxycurcumin, and Bisdemethoxycurcumin. (2002). doi:10.1021/JF025506A
Guo L, Salt T E, Luong V, Wood N, Cheung W, Maas A, Ferrari G, Russo-Marie F, Sillito A M, Cheetham M E et al (2007) Targeting amyloid-beta in glaucoma treatment. Proc Natl Acad Sci USA 104: 13444-13449 Doi 10.1073/pnas 0703707104
Guo L, Normando E M, Nizari S, Lara D, Cordeiro M F (2010) Tracking longitudinal retinal changes in experimental ocular hypertension using the cSLO and spectral domain-OCT. Investigative ophthalmology & visual science 51: 6504-6513 Doi 10.1167/iovs.10-5551
Jahoor A, Patel R, Bryan A, Do C, Frier J, Watters C, Wahli W, Li G, Williams S C, Rumbaugh K P (2008) Peroxisome Proliferator-Activated Receptors Mediate Host Cell Proinflammatory Response to Pseudomonda aeruginosa Autoinducer. Journal of Bacteriology 13: 4408-4415
Karlsson T, Turkina M, Yakymenkko, Magnusson K E, Vikstrom E (2012) The Pseudomonas aeruginosa N-Acylhomoserine Lactone Quorum Sensing Molecules Target IQGAP1 and Modulate Epithelial Cell Migration PLoS Pathogens 8, e1002953
Levkovitch-Verbin, H. et al. A model to study differences between primary and secondary degeneration of retinal ganglion cells in rats by partial optic nerve transection. IOVS 44, 3388-93. (2003).
Mayer L D, Hope M J, Cullis P R (1986) Vesicles of variable sizes produced by a rapid extrusion procedure. Biochim Biophys Acta 858: 161-168
Morrison, J. C. et al. A rat model of chronic pressure-induced optic nerve damage. Exp. Eye Res. 64, 85-96 (1997).
Nóbrega A M, Alves E N, Presgrave R F, Coste R N, Delgado I F. Determination of eye Irritation Potential of Low-irritant Products: Comparison of in vitro. Results with the in vivo Draize Rabbit Test. Brazilian Archives of Biology and Technology. 2012 (55): 381-388.
Normando E M, Davis B M, De Groef L, Nizari S, Turner L A, Ravindran N, Pahlitzsch M, Brenton J, Malaguamera, Guo L, Somavarapu S, Cordeiro M F (2016) Acta Neuropathologica Communications 4:86
Nucci, C. et al. Retinal damage caused by high intraocular pressure-induced transient ischemia is prevented by coenzyme Q10 in rat. Int. Rev. Neurobiol. 82, 397-406 (2007).
Reichl S (2008) Cell culture models of the human cornea—a comparative evaluation of their usefulness to determine ocular drug absorption in-vitro. J. Pharm. Pharmacol. 60, 299 Sigurdsson, H. H., Konrá\check{o}sdóttir, F., Loftsson, T. & Stefansson, E. Topical and systemic absorption in delivery of dexamethasone to the anterior and posterior segments of the eye. Acta Ophthalmol. Scand. 85, 598-602 (2007).
M. Warren, K. Atkinson, S. Steer, INVITTOX: The ERGATT/FRAME data bank of in vitro techniques in toxicology, Toxicol. Vitro 4 (1990) 707-710, http://dx.doi.org/10.1016/0887-2333(90)90148-M
Wang, W. et al. Enhanced bioavailability and efficiency of curcumin for the treatment of asthma by its formulation in solid lipid nanoparticles. Int. J. Nanomedicine 7, 3667-77 (2012).

1. A pharmaceutical composition comprising a peroxisome proliferator activated receptor (PPAR) modulator and an polymeric nanocarrier component, wherein the polymeric nanocarrier component is capable of solubilising the PPAR modulator in an aqueous medium and, wherein in the polymeric nanocarrier component is a micelle forming non-ionic surfactant.

2. A composition according to claim 1, wherein the micelle forming surfactant is selected from one or more of D-α-tocopherol polyethylene glycol 1000 succinate, PEGylated phospholipid derivatives (such as DSPE-PEG, DSPS-PEG, PLGA-PEG etc.), poloxamers (such as Lutrol F68, Lutrol F127 etc.), poly(lactic-co-glycolic acid) (PLGA), chitosan derivatives (chitosan-PEG etc.) or biodegradable polymer-PEG.

3. A composition according to claim 1 or 2, wherein the polymeric nanocarrier component further comprises one or more additional materials selected from ethylphosphatidylcholine and cationic lipids, such as N-[1-(2,3-Dioleoyloxy)propyl]-N,N,N-trimethylammonium methylsulfate (DOTAP), 18:1 DGS-NTA(Ni) [1,2-dioleoyl-sn-glycero-3-[(N-(5-amino-1-carboxypentyl)iminodiacetic acid)succinyl] or Solutol HS.

4. A composition according to any preceding claim, wherein the composition is in the form of a micellar composition.

5. A composition according to claim 4, wherein the micelles have a diameter of about 30 nm or less.

6. A composition according to any preceding claim, wherein the composition is in the form of a ternary system comprising an aqueous continuous phase, the PPAR modulator and polymeric nanocarrier component being predominantly present in a disperse phase distributed therein.

7. A composition according to any preceding claim which is sterile.

8. A composition according to any preceding claim, wherein the PPAR modulator is a PPAR-gamma agonist or a compound having PPAR-gamma agonist activity.

9. A composition according to any preceding claim, wherein the PPAR modulator is a thiazolidinedione, curcumin or resveratrol.

10. A composition according to claim 9, wherein the PPAR modulator is selected from one or more of pioglitazone, rosiglitazone, lobeglitazone, ciglitazone, darglitazone, englitazone, netoglitazone, rivoglitazone, Phytocannabinoid Δ9-THCA and troglitazone.

11. A composition according to any preceding claim, further comprising one or more pharmaceutically acceptable carriers or excipients.

12. A composition according to any preceding claim, which is suitable for non-parenteral delivery, e.g., topical delivery or oral delivery, or parenteral delivery.

13. A composition according to any preceding claim, which is suitable for intranasal delivery.

14. A composition according to any preceding claim for use in therapy.

15. A composition according to any preceding claim for use in the treatment or prevention of a CNS disorder.

16. A composition for use according to claim 15, wherein the CNS disorder is a neurodegenerative disorder, a retinal disorder or a brain disorder.

17. A composition for use according to claim 16, wherein the neurodegenerative condition is Parkinson's Disease, Alzheimer's Disease or Huntington's Disease.

18. A composition for use according to any of claims 15 to 17, wherein the composition is to be administered topically, such as ocularly, or intranasally.

19. A method for treating a CNS disorder, the method comprising administering a composition according to any of claims 1 to 13 to a patient.

20. The method of claim 19, wherein the CNS disorder is a neurodegenerative disorder, a retinal disorder or a brain disorder.

21. The method of claim 20, wherein the neurodegenerative condition is Parkinson's Disease, Alzheimer's Disease or Huntington's Disease.

22. The method of any of claims 19 to 21, wherein the composition is administered topically, such as ocularly, intranasally or dermally.

23. A method for preparing a PPAR agonist composition according to any of claims 1 to 13, the method comprising: (i) dissolving one or more polymeric nanocarrier components in a first solvent mixture; (ii) dissolving a PPAR modulator in a second solvent mixture; (iii) combining the dissolved polymeric nanocarrier component and dissolved PPAR modulator and drying the combination to a form a film; (iv) rehydrating the film with buffer to form a micellar solution; (v) sonicating the micellar solution to form a suspension; (vi) stabilising the suspension; and (vii) filtering the suspension to remove unencapsulated PPAR modulator.

24. The method of claim 23 wherein the first solvent mixture is ethanol.

25. The method of claim 23 or 24, wherein the first and second solvent mixtures are the same.

26. A composition as hereinbefore described with reference to the examples.
